(19) United States
(12) Patent Application Publication
(10) Pub. No.: US 2024/0408042 A1
(43) Pub. Date: Dec. 12, 2024

(54) ORALLY DISINTEGRATING BACLOFEN COMPOSITIONS AND METHODS OF MAKING SAME

(71) Applicant: Clinical Research Associates LLC, New York, NY (US)

(72) Inventors: John Amedio, Franklin, MA (US); David M. Lanzetta, Westborough, MA (US)

(21) Appl. No.: 18/331,693

(22) Filed: Jun. 8, 2023

(51) Int. Cl. A61K 31/197; A61K 9/20

(52) U.S. Cl. CPC A61K 31/197; A61K 9/2009; A61K 9/2018; A61K 9/2027; A61K 9/2054; A61K 9/2059

(57) ABSTRACT

This disclosure provides a novel orally disintegrating baclofen composition. The disclosed composition is stable at room temperature for at least 48 months. It maintains an excellent disintegration and dissolution profile and mouth feel after storage for an extended period of time.

### FIELD OF THE INVENTION

This invention relates generally to orally disintegrating baclofen compositions and methods of preparation thereof.

### BACKGROUND OF THE INVENTION

Mental retardation, Down's syndrome, fragile X syndrome, and autism are developmental and genetic disorders that affect day-to-day functioning, including learning, memory, speech, social skills, and behavior. Currently, available treatment regimens for humans with mental retardation, Down's syndrome, or fragile X syndrome to assist in day-to-day functioning include behavioral modifications and treatment with a range of medications, including anti-depressant and anti-psychotic drugs. However, such regimens frequently are not effective or may produce undesirable side-effects with long term use, particularly the use of anti-psychotic drugs.

(±)-4-Amino-3-(4-chlorophenyl) butanoic acid (baclofen), an analog of gamma-aminobutyric acid (i.e., GABA) that selectively activates GABAB receptors, has been used to treat mental retardation, Down's syndrome, fragile X syndrome, and autism. Baclofen, a zwitterionic amino acid, lacks the requisite physicochemical characteristics for effective passive permeability across cellular membranes. Accordingly, baclofen is a substrate for active transport mechanisms shared by neutral α-amino acids and β-amino acids (van Bree et al., Pharm. Res. 1988, 5 (6), 369-71). Baclofen is poorly absorbed following administration into the colon in animal models (Merino et al., Biopharm. Drug. Disp. 1989, 10 (3), 279-97), presumably because the transporter proteins mediating baclofen absorption in the upper region of the small intestine are not expressed in the large intestine.

Thus, there is a need to develop new, improved and effective baclofen formulations to achieve a better therapeutic profile of baclofen therapy.

### SUMMARY OF THE INVENTION

This disclosure addresses the need mentioned above in a number of aspects. In one aspect, this disclosure provides a novel orally disintegrating baclofen composition. The disclosed composition remains stable and maintains an excellent disintegration and dissolution profile after storage for an extended period of time (e.g., at least 48 months).

In some embodiments, the composition has at least one or more characteristics of: (a) disintegrating in less than 15 seconds upon admixing with the aqueous medium after storage for 64 months; (b) dissolving in less than 5 minutes upon admixing with the aqueous medium after storage for 64 months; (c) stable for at least 64 months under a storage condition at 25° C.±2° C./60% RH or at least 6 months under a storage condition at 40° C.±2° C./75% RH; and (d) generating less than 0.25% w/w of impurity 4-(4-chlorophenyl) pyrrolidin-2-one after storage at 25° C.±2° C./60% RH for 48 months or at 40° C.±2° C./75% RH for 6 months.

In some embodiments, the composition comprises baclofen, a stereoisomer thereof, a derivative thereof, an analog thereof, a prodrug thereof, or a pharmaceutically acceptable salt thereof; an excipient; a disintegrating agent; and a lubricating agent.

In some embodiments, baclofen is R-baclofen. In some embodiments, the excipient comprises F-MELT. In some embodiments, the disintegrating agent comprises crospovidone. In some embodiments, the lubricating agent comprises sodium stearyl fumarate.

In some embodiments, the composition further comprises a sweetener. In some embodiments, the sweetener comprises sucralose.

In some embodiments, the composition further comprises a flavoring agent, a masking agent, or a combination thereof. In some embodiments, the flavoring agent comprises a strawberry flavoring agent. In some embodiments, the masking agent comprises a second flavoring agent or a second sweetener.

In some embodiments, the composition comprises R-baclofen, F-MELT, crospovidone, sucralose, sodium stearyl fumarate, a flavoring agent, and a masking agent.

In some embodiments, the composition comprises: from 1.197% w/w to 1.463% w/w of R-baclofen, from 80.343% w/w to 98.197% w/w of F-MELT, from 4.5% w/w to 5.5% w/w of crospovidone, from 1.8% w/w to 2.2% w/w of sodium stearyl fumarate, from 1.8% w/w to 2.2% w/w of sucralose, from 0.27% w/w to 0.33% w/w of the flavoring agent, and from 0.09% w/w to 0.11% w/w of the masking agent.

In some embodiments, the composition comprises: about 1.33% w/w of R-baclofen, about 89.27% w/w of F-MELT, about 5% w/w of crospovidone, about 2% w/w of sodium stearyl fumarate, about 2% w/w of sucralose, about 0.3% w/w of the flavoring agent, and from 0.1% w/w of the masking agent.

In some embodiments, the composition comprises: about 1.33% w/w of R-baclofen, about 89.27% w/w of F-MELT type C, about 5% w/w of crospovidone, about 2% w/w of sodium stearyl fumarate, about 2% w/w of sucralose, about 0.3% w/w of the flavoring agent, and from 0.1% w/w of the masking agent.

In some embodiments, the composition comprises: about 2 mg of R-baclofen, about 133.9 mg of F-MELT (e.g., F-MELT type C), about 7.5 mg of crospovidone, about 3 mg of sodium stearyl fumarate, about 3 mg of sucralose, about 0.45 mg of the flavoring agent, and about 0.15 mg of the masking agent.

In some embodiments, the composition comprises: from 3% w/w to 3.67% w/w of R-baclofen, from 78.54% w/w to 95.99% w/w of F-MELT, from 4.5% w/w to 5.5% w/w of crospovidone, from 1.8% w/w to 2.2% w/w of sodium stearyl fumarate, from 1.8% w/w to 2.2% w/w of sucralose, from 0.27% w/w to 0.33% w/w of the flavoring agent, and from 0.09% w/w to 0.11% w/w of the masking agent.

In some embodiments, the composition comprises: about 3.33% w/w of R-baclofen, about 87.27% w/w of F-MELT type C, about 5% w/w of crospovidone, about 2% w/w of sodium stearyl fumarate, about 2% w/w of sucralose, about 0.3% w/w of the flavoring agent, and about 0.1% w/w of the masking agent.

In some embodiments, the composition comprises: about 5 mg of R-baclofen, about 130.9 mg of (e.g., F-MELT type C), about 7.5 mg of crospovidone, about 3 mg of sodium stearyl fumarate, about 3 mg of sucralose, about 0.45 mg of the flavoring agent, and about 0.15 mg of the masking agent.

In some embodiments, the composition comprises: from 6.0% w/w to 7.34% w/w of R-baclofen, from 75.54% w/w to 92.32% w/w of F-MELT, from 4.5% w/w to 5.5% w/w of crospovidone, from 1.8% w/w to 2.2% w/w of sodium stearyl fumarate, from 1.8% w/w to 2.2% w/w of sucralose, from 0.27% w/w to 0.33% w/w of the flavoring agent, and from 0.09% w/w to 0.11% w/w of the masking agent.

In some embodiments, the composition comprises: about 6.67% w/w of R-baclofen, about 83.93% w/w of F-MELT, about 5% w/w of crospovidone, about 2% w/w of sodium stearyl fumarate, about 2% w/w of sucralose, about 0.3% w/w of the flavoring agent, and about 0.1% w/w of the masking agent.

In some embodiments, the composition comprises: about 6.67% w/w of R-baclofen, about 83.93% w/w of F-MELT type C, about 5% w/w of crospovidone, about 2% w/w of sodium stearyl fumarate, about 2% w/w of sucralose, about 0.3% w/w of the flavoring agent, and about 0.1% w/w of the masking agent.

In some embodiments, the composition comprises: about 10 mg of R-baclofen, about 125.9 mg of F-MELT (e.g., F-MELT type C), about 7.5 mg of crospovidone, about 3 mg of sodium stearyl fumarate, about 3 mg of sucralose, about 0.45 mg of the flavoring agent, and about 0.15 mg of the masking agent.

In some embodiments, the composition comprises: from 9% w/w to 11% w/w of R-baclofen, from 72.54% w/w to 88.66% w/w of F-MELT, from 4.5% w/w to 5.5% w/w of crospovidone, from 1.8% w/w to 2.2% w/w of sodium stearyl fumarate, from 1.8% w/w to 2.2% w/w of sucralose, from 0.27% w/w to 0.33% w/w of the flavoring agent, and from 0.09% w/w to 0.11% w/w of the masking agent.

In some embodiments, the composition comprises: about 10% w/w of R-baclofen, about 80.6% w/w of F-MELT, about 5% w/w of crospovidone, about 2% w/w of sodium stearyl fumarate, about 2% w/w of sucralose, about 0.3% w/w of the flavoring agent, and about 0.1% w/w of the masking agent.

In some embodiments, the composition comprises: about 10% w/w of R-baclofen, about 80.6% w/w of F-MELT type C, about 5% w/w of crospovidone, about 2% w/w of sodium stearyl fumarate, about 2% w/w of sucralose, about 0.3% w/w of the flavoring agent, and about 0.1% w/w of the masking agent.

In some embodiments, the composition comprises: about 15 mg of R-baclofen, about 120.9 mg of F-MELT (e.g., F-MELT type C), about 7.5 mg of crospovidone, about 3 mg of sodium stearyl fumarate, about 3 mg of sucralose, about 0.45 mg of the flavoring agent, and about 0.15 mg of the masking agent.

In some embodiments, the composition is provided as one or more unit doses.

In another aspect, this disclosure also provides a unit dose comprising the composition as described herein.

In some embodiments, the unit dose is provided in a form of a tablet. In some embodiments, the tablet comprises 2 mg, 5 mg, 10 mg, or 15 mg of the baclofen, a stereoisomer thereof, a derivative thereof, an analog thereof, a prodrug thereof, or a pharmaceutically acceptable salt thereof.

In some embodiments, the tablet comprises 150 mg of the composition described herein. In some embodiments, the unit dose further comprises a foil packaging encapsulating the composition.

In yet another aspect, this disclosure further provides a method for forming a composition described herein. In some embodiments, the method comprises: (a) passing an active pharmaceutical ingredient through a 80 mesh screen, wherein the active pharmaceutical ingredient comprises baclofen, a stereoisomer thereof, a derivative thereof, an analog thereof, a prodrug thereof, or a pharmaceutically acceptable salt thereof; (b) blending about 0.2 kg of the active pharmaceutical ingredient and about 0.681 kg of F-MELT for about 10 minutes at about 25 rpm to obtain a first mixture; (c) blending about 1.834 kg of F-MELT for about 10 minutes at about 25 rpm; (d) adding to F-MELT of step (c) to the first mixture to obtain a second mixture, and passing the second mixture through a 50 mesh screen; (e) blending the second mixture with about 0.3 kg of the disintegrating agent, about 0.12 kg of the sweetener, about 0.018 kg of the flavoring agent, and about 0.006 kg of the masking agent for about 20 minutes at about 25 rpm to obtain a third mixture; and (f) passing the lubricating agent through a 30 mesh screen, and blending about 0.12 kg of the lubricating agent with the third mixture for about 5 minutes at about 25 rpm to obtain the composition.

In some embodiments, F-MELT comprises F-MELT. In some embodiments, the disintegrating agent comprises crospovidone. In some embodiments, the lubricating agent comprises sodium stearyl fumarate. In some embodiments, the sweetener comprises sucralose.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### DETAILED DESCRIPTION OF THE INVENTION

This disclosure provides a novel orally disintegrating baclofen composition. The disclosed composition is stable at room temperature for at least 48 months. It maintains an excellent disintegration and dissolution profile and mouth feel after storage for an extended period of time (e.g., at least 48 months).

### Baclofen Compositions

In some embodiments, the composition comprises a pharmaceutically active ingredient (API) that comprises baclofen, a stereoisomer thereof, a derivative thereof, an analog thereof, a prodrug thereof, or a pharmaceutically acceptable salt thereof. In some embodiments, baclofen is R-baclofen.

R-baclofen is known to be more potent than S-baclofen in reducing obsessive-compulsive and repetitive behavior, and in reducing audiogenic seizures, with minimal side effects. R-baclofen may be useful for the management of typical problem behavior, such as irritability and aggression, in humans with fragile X syndrome. Baclofen may improve irritable aberrant behavior and also have an improved safety and tolerability profile over atypical antipsychotics in the treatment of fragile X syndrome and/or autism. Baclofen may be used in combination with other medications, such as risperidone, antipsychotics, Group I mGluR antagonists and M1 muscarinic receptor antagonists.

In some embodiments, the composition has one or more characteristics of:
(a) disintegrating in less than 30 seconds (e.g., 30, 26, 22, 18, 16, 15, 14, 13, 12, 11, 10, 9, 8, 7, 6, 5, 4, 3, 2 seconds or less) upon admixing with the aqueous medium after storage for an extended period of time (e.g., 24, 28, 32, 36, 40, 44, 48, 52, 56, 58, 60, 62, 64 months or more);
(b) dissolving in less than 5 minutes (e.g., 5, 4, 3, 2, 1 minutes or less) upon admixing with the aqueous medium after storage for an extended period of time (e.g., 24, 28, 32, 36, 40, 44, 48, 52, 56, 58, 60, 62, 64 months or more);
(c) stable for at least 48 months (e.g., 48, 52, 56, 58, 60, 62, 64 months or more) under a storage condition at 25° C.±2° C./60% RH or at least 6 months (e.g., 6, 8, 10, 12, 14, 16 month or more) under a storage condition at 40° C.±2° C./75% RH; and
(d) generating less than 0.25% w/w of impurity 4-(4-chlorophenyl) pyrrolidin-2-one after storage at 25° C.±2° C./60% RH for 48 months or at 40° C.±2° C./75% RH for 6 months (e.g., 6, 8, 10, 12, 14, 16 month or more).

The composition may be formulated using conventional pharmaceutically acceptable carriers or excipients and well-established techniques. Without being limited thereto, such conventional carriers or excipients include diluents, binders and adhesives (i.e., cellulose derivatives and acrylic derivatives), lubricating agents (i.e., magnesium or calcium stearate, or vegetable oils, polyethylene glycols, talc, sodium lauryl sulfate, polyoxyethylene monostearate), thickeners, solubilizers, humectants, disintegrants, colorants, flavorings, stabilizing agents, sweeteners, and miscellaneous materials such as buffers and adsorbents in order to prepare a particular pharmaceutical composition. The stabilizing agents may include preservatives and antioxidants, amongst other components which will be readily apparent to one of ordinary skill in the art.

In some embodiments, the composition further comprises an excipient, a disintegrating agent, and/or a lubricating agent. In some embodiments, the composition further comprises a sweetener, a flavoring agent, a masking agent, or a combination thereof.

In some embodiments, the composition comprises from 1.197% w/w to 1.463% w/w (e.g., 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46% (w/w)) of R-baclofen.

In some embodiments, the composition comprises from 80.343% w/w to 98.197% w/w (e.g., 80.84%, 81.34%, 81.84%, 82.34%, 82.84%, 83.34%, 83.84%, 84.34%, 84.84%, 85.34%, 85.84%, 86.34%, 86.84%, 87.34%, 87.84%, 88.34%, 88.84%, 89.34%, 89.84%, 90.34%, 90.84%, 91.34%, 91.84%, 92.34%, 92.84%, 93.34%, 93.84%, 94.34%, 94.84%, 95.34%, 95.84%, 96.34%, 96.84%, 97.34%, 97.84%, 98% (w/w)) of the excipient.

In some embodiments, the composition comprises from 4.5% w/w to 5.5% w/w (e.g., 4.50%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.50% (w/w)) of the disintegrating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 1.33% w/w of R-baclofen, about 89.27% w/w of the excipient, about 5% w/w of the disintegrating agent, about 2% w/w of the lubricating agent, about 2% w/w of the sweetener, about 0.3% w/w of the flavoring agent, and from 0.1% w/w of the masking agent.

In some embodiments, the composition comprises: about 2 mg of R-baclofen, about 133.9 mg of the excipient, about 7.5 mg of the disintegrating agent, about 3 mg of the lubricating agent, about 3 mg of the sweetener, about 0.45 mg of the flavoring agent, and about 0.15 mg of the masking agent.

In some embodiments, the composition comprises from 3% w/w to 3.67% w/w (e.g., 3.00%, 3.05%, 3.10%, 3.15%, 3.20%, 3.25%, 3.30%, 3.35%, 3.40%, 3.45%, 3.50%, 3.55%, 3.60%, 3.65% (w/w)) of R-baclofen.

In some embodiments, the composition comprises from 78.54% w/w to 95.99% w/w (e.g., 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%, 89.04%, 89.54%, 90.04%, 90.54%, 91.04%, 91.54%, 92.04%, 92.54%, 93.04%, 93.54%, 94.04%, 94.54%, 95.04%, 95.54%, 95.99% (w/w)) of the excipient.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 3.33% w/w of R-baclofen, about 87.27% w/w of the excipient, about 5% w/w of the disintegrating agent, about 2% w/w of the lubricating agent, about 2% w/w of the sweetener, about 0.3% w/w of the flavoring agent, and about 0.1% w/w of the masking agent.

In some embodiments, the composition comprises: about 5 mg of R-baclofen, about 130.9 mg of the excipient, about 7.5 mg of the disintegrating agent, about 3 mg of the lubricating agent, about 3 mg of the sweetener, about 0.45 mg of the flavoring agent, and about 0.15 mg of the masking agent.

In some embodiments, the composition comprises from 6.0% w/w to 7.34% w/w (e.g., 6.0%, 6.1%, 6.2%, 6.3%, 6.4%, 6.5%, 6.6%, 6.7%, 6.8%, 6.9%, 7.0%, 7.1%, 7.2%, 7.3% (w/w)) of R-baclofen.

In some embodiments, the composition comprises from 75.54% w/w to 92.32% w/w (e.g., 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%, 89.04%, 89.54%, 90.04%, 90.54%, 91.04%, 91.54%, 92.04% (w/w)) of the excipient.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 6.67% w/w of R-baclofen, about 83.93% w/w of the excipient, about 5% w/w of the disintegrating agent, about 2% w/w of the lubricating agent, about 2% w/w of the sweetener, about 0.3% w/w of the flavoring agent, and about 0.1% w/w of the masking agent.

In some embodiments, the composition comprises: about 10 mg of R-baclofen, about 125.9 mg of the excipient, about 7.5 mg of the disintegrating agent, about 3 mg of the lubricating agent, about 3 mg of the sweetener, about 0.45 mg of the flavoring agent, and about 0.15 mg of the masking agent.

In some embodiments, the composition comprises from 9% w/w to 11% w/w (e.g., 9%, 9.2%, 9.4%, 9.6%, 9.8%, 10.0%, 10.2%, 10.4%, 10.6%, 10.8%, 11% (w/w)) of R-baclofen.

In some embodiments, the composition comprises from 72.54% w/w to 88.66% w/w (e.g., 72.54%, 73.04%, 73.54%, 74.04%, 74.54%, 75.04%, 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54% (w/w)) of the excipient.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 10% w/w of R-baclofen, about 80.6% w/w of the excipient, about 5% w/w of the disintegrating agent, about 2% w/w of the lubricating agent, about 2% w/w of the sweetener, about 0.3% w/w of the flavoring agent, and about 0.1% w/w of the masking agent.

In some embodiments, the composition comprises: about 15 mg of R-baclofen, about 120.9 mg of the excipient, about 7.5 mg of the disintegrating agent, about 3 mg of the lubricating agent, about 3 mg of the sweetener, about 0.45 mg of the flavoring agent, and about 0.15 mg of the masking agent.

### Baclofen

Baclofen is referred to as Baclon, Lioresal, Kemstro, and Myospan. Baclofen is also referred to as β-(Aminomethyl)-4-chlorobenzenepropanoic acid; β-(aminomethyl)-p-chlorohydrocinnamic acid; γ-amino-β-(p-chlorophenyl) butyric acid; B-(4-chlorophenyl) GABA. Baclofen has a molecular formula of C₁₀H₁₂ClNO₂ (molecular weight: 213.66 Dalton) and a chemical structure as follows:

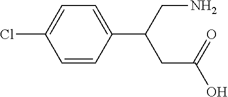 [SMILES: NCC(CC(=O)O)c1ccc(Cl)cc1]

In some embodiments, baclofen may exist in various forms, such as stereoisomers, racemates, derivatives, prodrugs, analogs, or pharmaceutically acceptable salts, that are also suitable for use in the disclosed methods.

“Isomers” are different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either (R) or(S). Resolved compounds whose absolute configuration is unknown can be designated (+) or (−) depending on the direction (dextro- or levorotatory) in which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or(S). The present chemical entities, pharmaceutical compositions, and methods are meant to include all such possible isomers, including racemic mixtures, optically pure forms, and intermediate mixtures. Optically active (R)- and(S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related to mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

“Racemate” or “racemic mixture” means a compound of equimolar quantities of two enantiomers, wherein such mixtures exhibit no optical activity, i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R.” “S.” “St.” “R*.” “E.” “Z.” “cis.” and “trans” indicate configurations relative to the core molecule.

A “derivative,” as used herein, refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing, directly or indirectly, the activity disclosed herein.

An “analog” refers to a small organic compound, a nucleotide, a protein, or a polypeptide that possesses similar or identical activity or function(s) as the compound, nucleotide, protein or polypeptide or compound having the desired activity of this disclosure, but need not necessarily include a sequence or structure that is similar or identical to the sequence or structure of the preferred embodiments.

A “prodrug” refers to a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility, or delayed release in a mammalian organism (see, e.g., Bundgaard, H., Design of Prodrugs (1985) (Elsevier, Amsterdam). The term “prodrug” also refers to any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino, or free mercapto group, respectively. Examples of prodrugs include, but are not limited to, acetates, formates, and benzoate derivatives of alcohol, various ester derivatives of a carboxylic acid, or acetamide, formamide, and benzamide derivatives of an amine functional group in the active compound. Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pgs 113-191 (Harwood Academic Publishers, 1991); and (d) Hydrolysis in Drug and Prodrug Metabolism, Bernard Testa and Joachim M. Mayer, (Wiley-VCH, 2003).

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable salt” refers to a salt of the administered compounds prepared from pharmaceutically acceptable non-toxic acids, including inorganic acids, organic acids, solvates, hydrates, or clathrates thereof.

### Excipients

In some embodiments, the composition comprises one or more pharmaceutically acceptable excipients, including fillers, diluents and/or binders. Non-limiting examples of suitable fillers, diluents and/or binders include lactose (e.g., spray-dried lactose, α-lactose, β-lactose), microcrystalline cellulose, hydroxypropyl cellulose, hydroxypropyl methylcellulose (HPMC), methylcellulose, hydroxyethyl cellulose, sodium carboxymethylcellulose, carboxymethylene, carboxymethyl hydroxyethyl cellulose and other cellulose derivatives, sucrose, agarose, sorbitol, mannitol, dextrins, maltodextrins, starches or modified starches (including potato starch, maize starch, and rice starch), calcium phosphate (e.g., basic calcium phosphate, calcium hydrogen phosphate, dicalcium phosphate hydrate), calcium sulfate, calcium carbonate, and potassium hydrogen phosphate.

Non-limiting examples of diluents include, but are not limited to, calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, and sugar.

Non-limiting examples of binders include, but are not limited to, acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, maltodextrin, and pregelatinized starch.

In some embodiments, the excipient comprises F-MELT. F-MELT is a premix of carbohydrates, disintegrants, and inorganic ingredients. F-MELT type C comprises D-Mannitol, Xylitol, microcrystalline cellulose (MCC), crospovidone, and dibasic calcium phosphate anhydrous (DCPA). F-MELT type M comprises D-Mannitol, Xylitol, microcrystalline cellulose (MCC), crospovidone, and magnesium aluminometasilicate (MAS).

In some embodiments, the composition comprises 30% w/w to 99% w/w (e.g., 30%, 31%, 31%, 32%, 32%, 33%, 33%, 34%, 34%, 35%, 35%, 36%, 36%, 37%, 37%, 38%, 38%, 39%, 39%. 40%, 40%, 41%, 41%, 42%, 42%, 43%, 43%, 44%, 44%, 45%, 45%, 46%, 46%, 47%, 47%, 48%, 48%, 49%, 49%, 50%, 50%, 51%, 51%, 52%, 52%, 53%, 53%, 54%, 54%, 55%, 55%, 56%, 56%, 57%, 57%, 58%, 58%, 59%, 59%, 60%, 60%, 62%, 64%, 66%, 68%, 70%, 72%, 74%, 76%, 78%, 80%, 82%, 84%, 86%, 88%, 90%, 92%, 94%, 96%, 98%, 99% (w/w)) of one or more excipients, such as F-MELT (e.g., F-MELT type C or F-MELT type M).

In some embodiments, the composition comprises about 70% w/w to about 99% w/w (e.g., 70.0%, 70.5%, 71.0%, 71.5%, 72.0%, 72.5%, 73.0%, 73.5%, 74.0%, 74.5%, 75.0%, 75.5%, 76.0%, 76.5%, 77.0%, 77.5%, 78.0%, 78.5%, 79.0%, 79.5%, 80.0%, 80.5%, 81.0%, 81.5%, 82.0%, 82.5%, 83.0%, 83.5%, 84.0%, 84.5%, 85.0%, 85.5%, 86.0%, 86.5%, 87.0%, 87.5%, 88.0%, 88.5%, 89.0%, 89.5%, 90.0%, 90.5%, 91.0%, 91.5%, 92.0%, 92.5%, 93.0%, 93.5%, 94.0%, 94.5%, 95.0%, 95.5%, 96.0%, 96.5%, 97.0%, 97.5%, 98.0%, 98.5%, 99.0% (w/w)) of F-MELT type C.

### Disintegrating Agent

A disintegrant suitable for incorporation into the disclosed orally disintegrating compositions includes, without limitation, a cross-linked polyvinylpyrrolidone (referred to as polyplasdone or crospovidone (Polyplasdone XL-10)), a cross-linked sodium carboxymethyl cellulose (referred to as croscarmellose sodium), hydroxypropyl methylcellulose (HPMC), hydroxypropyl cellulose (HPC), low-substituted hydroxypropyl cellulose (L-HPC), alginic acid, sodium starch glycolate, starch (e.g., pregelatinized starch), microcrystalline cellulose, powdered cellulose, starch, sodium alginate, and a mixture thereof.

In some embodiments, the disintegrating agent comprises crospovidone. In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of crospovidone.

### Lubricating Agents

Non-limiting examples of lubricants or glidants) may include stearic acid, magnesium stearate, calcium stearate, zinc stearate, or other metallic stearate, talc, waxes and glycerides, sterotex, polyoxyethylene monostearate, talc, light mineral oil, polyethylene glycol, glyceryl behenate, colloidal silica, hydrogenated vegetable oils, corn starch, sodium stearyl fumarate, polyethylene glycols, alkyl sulfates, sodium benzoate, and sodium acetate, sodium lauryl sulfate, magnesium lauryl sulfate, and light mineral oil.

In some embodiments, the lubricating agent comprises sodium stearyl fumarate. In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of sodium stearyl fumarate.

### Sweeteners

As used herein, the term “sweetener” includes all artificial and natural sweeteners, sugar alcohols (or polyols) and sugar sweeteners (or carbohydrates). Artificial and natural sweeteners include but are not limited to abiziasaponin, abrusosides, in particular abrusoside A, abrusoside B, abrusoside C, abrusoside D, acesulfame potassium, advantame, albiziasaponin, alitame, aspartame, superaspartame, bayunosides, in particular bayunoside 1, bayunoside 2, brazzein, bryoside, bryonoside, bryonodulcoside, carnosifloside, carrelame, curculin, cyanin, chlorogenic acid, cyclamates and its salts, cyclocaryoside I, dihydroquercetin-3-acetate, dihydroflavenol, dulcoside, gaudichaudioside, glycyrrhizin, glycyrrhetin acid, gypenoside, hematoxylin, hernandulcin, isomogrosides, in particular iso-mogroside V, lugduname, magap, mabinlins, micraculin, mogrosides (lo han guo), in particular mogroside IV and mogroside V, monatin and its derivatives, monellin, mukurozioside, naringin dihydrochalcone (NarDHC), neohesperidin dihydrochalcone (NDHC), neotame, osladin, pentadin, periandrin I-V, perillartine, D-phenylalanine, phlomisosides, in particular phlomisoside 1, phlomisoside 2, phlomisoside 3, phlomisoside 4, phloridzin, phyllodulcin, polpodiosides, polypodoside A, pterocaryosides, rebaudiosides, in particular rebaudioside A, rebaudioside B, rebaudioside C, rebaudioside D, rebaudioside F, rebaudioside G, rebaudioside H), rubusosides, saccharin and its salts and derivatives, scandenoside, selligucanin A, siamenosides, in particular siamenoside I, stevia, steviolbioside, stevioside and other steviol glycosides, strogines, in particular strogin 1, strogin 2, strogin 4, suavioside A, suavioside B, suavioside G, suavioside H, suavioside I, suavioside J, sucralose, sucronate, sucrooctate, talin, telosmoside A15, thaumatin, in particular thaumatin I and II, trans-anethol, trans-cinnamaldehyde, trilobtain and D-tryptophane, including extracts or enriched fractions of the natural sweeteners. Sugar alcohols (or polyols) include but are not limited to erythritol, galactitol, hydrogenated starch syrups including maltitol and sorbitol syrups, inositols, isomalt, lactitol, maltitol, mannitol, xylitol, and combinations thereof. Sugar sweeteners (or carbohydrates) include monosaccharides, disaccharides, oligosaccharides and polysaccharides such as but not limited to arabinose, dextrin, dextrose, fructose, high fructose corn syrup, fructooligosaccharides, fructooligosaccharide syrups, galactose, galactooligosaccharides, glucose, glucose and (hydrogenated) starch syrups/hydrolysates, isomaltulose, lactose, hydrolysed lactose, maltose, mannose, rhamnose, ribose, sucrose, stachyose, tagatose, trehalose, xylose, and combinations thereof. The sweeteners are known substances and are for example those described by H. Mitchell (H. Mitchell, “Sweeteners and Sugar Alternatives in Food Technology”, Backwell Publishing Ltd, 2006.) and in WO 2009/023975A2, each of which is incorporated herein by reference in its entirety. The above-identified sweeteners are known in the art and are commercially available.

Suitable hydrogenated starch hydrolysates include, but are not limited to, those disclosed in U.S. Pat. No. 4,279,931, which is hereby incorporated by reference, and various hydrogenated glucose syrups and/or powders which contain sorbitol, maltitol, hydrogenated disaccharides, hydrogenated higher polysaccharides, or combination thereof. Hydrogenated starch hydrosylates are primarily prepared by the controlled catalytic hydrogenation of con syrups. The resulting hydrogenated starch hydrosylates are mixtures of monomeric, dimeric, and polymeric saccharides. The hydrogenated starch hydrolysates are known in the art and are commercially available.

As used herein, the term “sweetness enhancer” means any compound capable of enhancing or intensifying the perception of sweet taste of sweetener compositions or sweetened compositions. The term “sweetness enhancer” is synonymous with the terms “sweet taste potentiator,” “sweetness potentiator,” and “sweetness intensifier.”

In some embodiments, the composition may include one or more sweeteners. In some embodiments, the composition may include a mixture of sweeteners. In some embodiments, the composition may include neotame.

The amount of sweetener presented in the composition should be sufficient to mask the taste of the pharmaceutically active ingredient and provide a sweet taste to the composition. The amount of sweetener presented in the composition depends on the sweetness intensity of selected sweeteners. For example, a lower amount of sweetener may be used when the sweetener intensity is higher.

In some embodiments, the sweetener comprises sucralose. In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of sucralose.

### Flavoring Agents

As used herein, the term “flavoring agents” may include those flavor ingredients known in the art, such as natural and artificial flavors. These flavoring agents may be chosen from synthetic flavor oils and flavoring ingredient aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also, useful flavoring agents can be artificial, natural, and synthetic fruit flavors, such as vanilla and citrus oils, including lemon, orange, lime, grapefruit, yazu, sudachi, and fruit essences, including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath-freshening properties, particularly the mint flavors when used in combination with cooling agents.

Other useful flavoring agents include aldehydes and esters such as cinnamyl acetate, cinnamaldehyde, citral diethylacetal, dihydrocarvyl acetate, eugenyl formate, p-methylamisol, and so forth may be used. Generally, any flavoring ingredient or food additive such as those described in Chemicals Used in Food Processing, publication 1274, pages 63-258, by the National Academy of Sciences, may be used. This publication is incorporated herein by reference.

Further examples of aldehyde flavoring agents include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavoring agents are merely exemplary and are not meant to limit either the term “flavoring ingredient” or the scope of the invention generally.

In some embodiments, the flavoring ingredient may be employed in either liquid form and/or dried form. When employed in the latter form, suitable drying means such as spray drying the oil may be used. Alternatively, the flavoring ingredient may be absorbed onto water soluble materials, such as cellulose, starch, sugar, maltodextrin, gum arabic and so forth or may be encapsulated. The actual techniques for preparing such dried forms are well-known.

In some embodiments, the flavoring agents may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

In some embodiments, the flavoring agent comprises a strawberry flavoring agent. In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent, e.g., a strawberry flavor.

### Additional Masking Agents

As used herein, the term “masking” as it relates to the composition X as defined above means that consumption thereof masks a perception of a taste, off-taste, aftertaste or lingering sweetness of a sweetener composition or a consumable product in the oral cavity. As used herein, the term “off-taste” means any taste of a sweetener, a sweetness enhancer or a consumable product, e.g., a food or beverage, that is perceived in the oral cavity on or after consumption thereof and that can stay there for a few minutes. Off-tastes include but are not limited to acidic, astringent, bitter, liquorice, metallic or throat-burning. In one embodiment, the off-taste is a metallic off-taste provided by neotame. As used herein, the term “aftertaste” means any taste of a sweetener, a sweetness enhancer or a consumable product, e.g., a food or beverage, that is perceived in the oral cavity after the sweetener, the sweetness enhancer or the consumable product is removed from the oral cavity, e.g., by swallowing or disgorging. The aftertaste may remain in the oral cavity for example, for a few minutes or a few hours. Unpleasant aftertastes include but are not limited to bitter and/or astringent aftertastes. In some embodiments, the aftertaste is provided by acesulfame potassium, saccharin, and/or stevioside. As used herein, the term “lingering sweetness” means a very long-lasting sweetening effect of a sweetener, a sweetness enhancer or a consumable product, e.g., a food or beverage, which is perceived in the oral cavity after the sweetener, the sweetness enhancer or the consumable product is removed from the oral cavity by swallowing or disgorging. The lingering sweetness may remain in the oral cavity for example, for a few minutes or a few hours.

The masking agents may include sweeteners and/or flavoring agents. In some embodiments, masking agents include one or more sweeteners or sweetness enhancers. Non-limiting examples of artificial or natural sweeteners for taste masking include but are not limited to abiziasaponin, abrusosides, in particular abrusoside A, abrusoside B, abrusoside C, abrusoside D, acesulfame potassium, advantame, albiziasaponin, alitame, aspartame, superaspartame,-bayunosides, in particular bayunoside 1, bayunoside 2, brazzein, bryoside, bryonoside, bryonodulcoside, carnosifloside, carrelame, curculin, cyanin, chlorogenic acid, cyclamates and its salts, cyclocaryoside I, dihydroquercetin-3-acetate, dihydroflavenol, dulcoside, gaudichaudioside, glycyrrhizin, glycyrrhetin acid, gypenoside, hematoxylin, hernandulcin, isomogrosides, in particular iso-mogroside V, lugduname, magap, mabinlins, micraculin, mogrosides (lo han guo), in particular mogroside IV and mogroside V, monatin and its derivatives, monellin, mulcurozioside, naringin dihydrochalcone (NarDHC), neohesperidin dihydrochalcone (NDHC), neotame, osladin, pentadin, periandrin I-V, perillartine, D-phenylalanine, phlomisosides, in particular phlomisoside 1, phlomisoside 2, phlomisoside 3, phlomisoside 4, phloridzin, phyllodulcin, polpodiosides, polypodoside A, pterocaryosides, rebaudiosides, in particular rebaudioside A, rebaudioside B, rebaudioside C, rebaudioside D, rebaudioside F, rebaudioside G, rebaudioside H), rubusosides, saccharin and its salts and derivatives, scandenoside, selligucanin A, siamenosides, in particular siamenoside I, stevia, steviolbioside, stevioside and other steviol glycosides, strogines, in particular strogin 1, strogin 2, strogin 4, suavioside A, suavioside B, suavioside G, suavioside H, suavioside I, suavioside J, sucralose, sucronate, sucrooctate, talin, telosmoside A15, thaumatin, in particular thaumatin I and II, trans-anethol, trans-cinnamaldehyde, trilobatin and D-tryptophane, including extracts or enriched fractions of the natural sweeteners.

Suitable sweetness enhancers are well known in the art. In one embodiment, the at least one sweetness enhancer may be selected from the group consisting of terpenes (such as sesquiterpenes, diterpenes, and triterpenes), flavonoids, amino acids, proteins, polyols, other known natural sweeteners (such as cinnamaldehydes, selligucains and hematoxylins), secodammarane glycosides, and analogues thereof.

Non-limiting examples of sweetness enhancers include stevioside, steviolbioside, rebaudioside A, rebaudioside B, rebaudioside C, rebaudioside D, rebaudioside F, dulcoside A, rubusoside; hernandulcin; pine rosin diperpenoid; mukurozioside; baiyunosdie; phlomisoside, such as phlomisoside I and phlomisodie II; glycyrrhizic acid; periandrins, such as periandrin I, periandrin II, periandrin III, and periandrin IV; osladin; polypodosides, such as polypodoside A and polypodoside B; mogrosides, such as mogroside IV and mogroside V; abrusoside A and abrusosdie B; cyclocariosdies, such as cyclocarioside A and cyclocarioside B; pterocaryoside A and pterocaryoside B; flavonoids, such as phyllodulcin, phloridzin, neoastilbin, and dihydroquercetin acetate; amino acids, such as glycine and monatin; proteins, such as thaumatins (thaumatin I, thaumatin II, thaumatin iii, and thaumatin IV), monellin, mabinlins (mabinlin I and mabinlin II), brazzein, miraculin, and curculin; polyols such as erythritol; cinnamaldehyde; selligueains, such as selligueain A and selligueain B; hematoxylin; and mixtures thereof.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises R-baclofen, F-MELT, crospovidone, sucralose, sodium stearyl fumarate, a flavoring agent, and a masking agent.

In some embodiments, the composition comprises from 1.197% w/w to 1.463% w/w (e.g., 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46% (w/w)) of R-baclofen

In some embodiments, the composition comprises from 80.343% w/w to 98.197% w/w (e.g., 80.84%, 81.34%, 81.84%, 82.34%, 82.84%, 83.34%, 83.84%, 84.34%, 84.84%, 85.34%, 85.84%, 86.34%, 86.84%, 87.34%, 87.84%, 88.34%, 88.84%, 89.34%, 89.84%, 90.34%, 90.84%, 91.34%, 91.84%, 92.34%, 92.84%, 93.34%, 93.84%, 94.34%, 94.84%, 95.34%, 95.84%, 96.34%, 96.84%, 97.34%, 97.84%, 98% (w/w)) of F-MELT.

In some embodiments, the composition comprises from 4.5% w/w to 5.5% w/w (e.g., 4.50%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.50% (w/w)) of crospovidone.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of stearyl fumarate, from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 1.33% w/w of R-baclofen, about 89.27% w/w of F-MELT, about 5% w/w of crospovidone, about 2% w/w of stearyl fumarate, about 2% w/w of the sweetener, about 0.3% w/w of the flavoring agent, and from 0.1% w/w of the masking agent.

In some embodiments, the composition comprises: about 1.33% w/w of R-baclofen, about 89.27% w/w of F-MELT type C, about 5% w/w of crospovidone, about 2% w/w of stearyl fumarate, about 2% w/w of the sweetener, about 0.3% w/w of the flavoring agent, and from 0.1% w/w of the masking agent.

In some embodiments, the composition comprises: about 2 mg of R-baclofen, about 133.9 mg of F-MELT, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises: about 2 mg of R-baclofen, about 133.9 mg of F-MELT type C, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises from 3% w/w to 3.67% w/w (e.g., 3.00%, 3.05%, 3.10%, 3.15%, 3.20%, 3.25%, 3.30%, 3.35%, 3.40%, 3.45%, 3.50%, 3.55%, 3.60%, 3.65% (w/w)) of R-baclofen.

In some embodiments, the composition comprises from 78.54% w/w to 95.99% w/w (e.g., 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%, 89.04%, 89.54%, 90.04%, 90.54%, 91.04%, 91.54%, 92.04%, 92.54%, 93.04%, 93.54%, 94.04%, 94.54%, 95.04%, 95.54%, 95.99% (w/w)) of F-MELT.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of crospovidone.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of stearyl fumarate.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 3.33% w/w of R-baclofen, about 87.27% w/w of F-MELT, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 3.33% w/w of R-baclofen, about 87.27% w/w of F-MELT type C, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 5 mg of R-baclofen, about 130.9 mg of F-MELT, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises: about 5 mg of R-baclofen, about 130.9 mg of F-MELT type C, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises from 6.0% w/w to 7.34% w/w (e.g., 6.0%, 6.1%, 6.2%, 6.3%, 6.4%, 6.5%, 6.6%, 6.7%, 6.8%, 6.9%, 7.0%, 7.1%, 7.2%, 7.3% (w/w)) of R-baclofen.

In some embodiments, the composition comprises from 75.54% w/w to 92.32% w/w (e.g., 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%, 89.04%, 89.54%, 90.04%, 90.54%, 91.04%, 91.54%, 92.04% (w/w)) of F-MELT.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of crospovidone.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of stearyl fumarate.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 6.67% w/w of R-baclofen, about 83.93% w/w of F-MELT, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 6.67% w/w of R-baclofen, about 83.93% w/w of F-MELT type C, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 10 mg of R-baclofen, about 125.9 mg of F-MELT, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises: about 10 mg of R-baclofen, about 125.9 mg of F-MELT type C, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises from 9% w/w to 11% w/w (e.g., 9%, 9.2%, 9.4%, 9.6%, 9.8%, 10.0%, 10.2%, 10.4%, 10.6%, 10.8%, 11% (w/w)) of R-baclofen.

In some embodiments, the composition comprises from 72.54% w/w to 88.66% w/w (e.g., 72.54%, 73.04%, 73.54%, 74.04%, 74.54%, 75.04%, 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54% (w/w)) of F-MELT.

In some embodiments, the composition comprises from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of crospovidone.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of stearyl fumarate.

In some embodiments, the composition comprises from 1.8% w/w to 2.2% w/w (e.g., 1.80%, 1.85%, 1.90%, 1.95%, 2.00%, 2.05%, 2.10%, 2.15%, 2.20% (w/w)) of the sweetener.

In some embodiments, the composition comprises from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent.

In some embodiments, the composition comprises from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: about 10% w/w of R-baclofen, about 80.6% w/w of F-MELT, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 10% w/w of R-baclofen, about 80.6% w/w of F-MELT type C, about 5% w/w of crospovidone, about 2% w/w of stearyl fumarate, about 2% w/w of the sweetener, about 0.3% w/w of the flavoring agent, and about 0.1% w/w of the masking agent.

In some embodiments, the composition comprises: about 15 mg of R-baclofen, about 120.9 mg of F-MELT, about 7.5 mg of crospovidone, about 3 mg of stearyl fumarate, about 3 mg of the sweetener, about 0.45 mg of the flavoring agent, and about 0.15 mg of the masking agent.

In some embodiments, the composition comprises: about 15 mg of R-baclofen, about 120.9 mg of F-MELT type C, about 7.5 mg of crospovidone, about 3 mg of stearyl fumarate, about 3 mg of the sweetener, about 0.45 mg of the flavoring agent, and about 0.15 mg of the masking agent.

In some embodiments, the composition may include an additional therapeutic agent. In some embodiments, the additional therapeutic agent is selected from the group consisting of a Group II mGluR agonist, Group II mGluR agonist, GSK3β antagonists, NAAG peptidase inhibitors, Group I mGluR antagonist, an antidepressant, an anti-psychotic, an α2-adrenergic agonist, an anticonvulsant, a nicotinic receptor agonist, an endocannabinoid receptor antagonist, a M1 muscarinic receptor antagonist, and an AMPA agonist.

In one embodiment, the Group I mGluR antagonist is a mGluR5 antagonist. In another embodiment, the Group I mGluR antagonist is an mGluR1 antagonist. Suitable Group I mGluR antagonists for use are described in U.S. Pat. Nos. 6,890,931 and 6,916,821, the teachings of both of which are hereby incorporated by reference in their entirety. Suitable Group I mGluR antagonists can include, for example, (E)-6-methyl-2-styryl-pyridine (SIB 1893), 6-methyl-2-phenylazo)-3-pyridinol, x-methyl-4-carboxyphenylglycine (MCPG) and 2-methyl-6-(phenylthynyl)-pyridine (MPEP).

Exemplary antagonists of mGluR5 for use in the methods of the invention in combination with GABA agonists, in particular GABA (B) agonists (e.g., baclofen), are described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, U.S. Pat. Nos. 6,218,385, 5,672,592, 5,795,877, 5,863,536, 5,880,112, 5,902,817, all of which are incorporated by reference in their entirety. Different classes of mGluR5 antagonists are described in WO 01/08705, WO 99/44639 and WO 98/34907, the teachings of all of which are hereby incorporated by reference in their entirety.

The antipsychotic compound employed in the methods of the invention can be a typical antipsychotic compound (also referred to as “a typical antipsychotic agent” or a “typical antipsychotic drug”). In another embodiment, the antipsychotic compound is an atypical antipsychotic compound (also referred to as an “atypical antipsychotic agent,” an “atypical antipsychotic drug” or a “second generation antipsychotic”).

Exemplary atypical antipsychotic compounds for use in the methods of the invention can be at least one member selected from the group consisting of zuclopenthixol, amisulpride, aripiprazole (7-[4-[4-(2,3-dichlorophenyl)-1-piperazinyl]butoxy]-3-4-dihydrocarbostyril), nemonapride, abaperidone (7-[3-[4-(6-fluoro-1,2-benzisoxazol-3-yl)-1-piperidinyl]propoxy]-3-(-hydroxymethyl)-4H-1-benzopyran-4-one, U.S. Pat. No. 5,736,588, the teachings of which are hereby incorporated by reference in its entirety; belaperidone ((1.alpha.,5.alpha.,6.alpha.)-3-[2-[6-(4-fluorophenyl)-3-azabicyclo[-3.2.0]-hept-3-yl]ethyl]-2,4 (1H,3H) quinazolinedione, U.S. Pat. No. 5,475,105, the teachings of which are hereby incorporated by reference in its entirety; clozapine (8-chloro-11-(4-methyl-1-piperazinyl)-5H-dibenzo[b,c][1,4]diazepine, U.S. Pat. No. 3,539,573, the teachings of which are hereby incorporated by reference in its entirety issued; iloperidone (1-[4-[3-[4-(6-fluoro-1,2-benzisoxazol-3-yl)-1-piperidinyl]-3-methoxy-phenyl]ethanone; EP-402,644, the teachings of which are hereby incorporated by reference in its entirety; olanzapine (2-methyl-4-(4-methyl-1-piperazinyl)-10H-thieno[2,3-b][1,5]benzodiazepine; U.S. Pat. No. 5,229,382, the teachings of which are hereby incorporated by reference in its entirety; perospirone (cis-2-[4-[4-(1,2-benzisothiazol-3-yl)-1-piperazinyl]butyl]-hexahydro-o-1H-isoindole-1,3 (2H)-dione, U.S. Pat. No. 4,745,117, the teachings of which are hereby incorporated by reference in its entirety; risperidone (3-[2-[4-(6-fluoro-1,2-benzisoxazol-3-yl) piperidino]ethyl]-2-methyl-6,7,8,9-tetrahydro-4H-pyrido[1,2-. alpha.]pyrimidin-4-onc), U.S. Pat. No. 4,804,663, the teachings of which are hereby incorporated by reference in its entirety; sertindole (1-[2-[4-[5-chloro-1-(4-fluorophenyl-1H-indol-3-yl]-1-piperidinyl]eth-yl]imidazolidin-2-one), U.S. Pat. Nos. 4,710,500; 5,112,838; and 5,238,945, the teachings of all of which are hereby incorporated by reference in their entirety; tiospirone (8-[4-[4-(1,2-benzisothiazol-3-yl)-1-piperazinyl]butyl]-8-azaspiro[4-0.5]decane-7,9-dione), U.S. Pat. No. 4,411,901, the teachings of which are hereby incorporated by reference in its entirety; ziprasidone (5-[2-[4-(1,2-benzoisothiazole-3-yl)-1-piperazinyl]ethyl]-6-chloro-1-,3-dihydro-2-one), U.S. Pat. No. 4,831,031, the teachings of which are hereby incorporated by reference in its entirety; zotepine (2-[(8-chlorodibenzo[b,f]thiepin-10-yl)oxy]-N,N-dimethyl-ethanamine), U.S. Pat. No. 3,704,245, the teachings of which are hereby incorporated by reference in its entirety; quetiapine (5-[2-(4-dibenzo[b,f][1,4]thiazepin-11-yl-1 pipcrazinyl) ethoxy]ethano-1), U.S. Pat. No. 4,879,288, the teachings of which are hereby incorporated by reference in its entirety; and blonanserin (2-(4-ethyl-1-piperazinyl)-4-(4-fluorophenyl)-5,6,7,8,9,10-hexahydro-1-cycloocta[b]pyridinc), U.S. Pat. No. 5,021,421, the teachings of which are hereby incorporated by reference in its entirety; 2002/0123490, the teachings of which are hereby incorporated by reference in its entirety).

Antipsychotic agents, including atypical antipsychotic compounds for use in the invention can include, for example, Acetophenazine Maleate; Alentemol Hydrobromide; Alpertine; Azaperone; Batelapine Malcate; Benperidol; Benzindopyrine Hydrochloride; Brofoxine; Bromperidol; Butaclamol Hydrochloride; Butaperazine; Carphenazine Maleate; Carvotroline Hydrochloride; Chlorpromazine; Chlorprothixene; Cinperene; Cintriamide; Clomacran Phosphate; Clopenthixol; Clopimozide; Clopipazan Mesylate; Cloroperone Hydrochloride; Clothiapine; Clothixamide Maleate; Clozapine; Cyclophenazine Hydrochloride; properidol; Etazolate Hydrochloride; Fenimide; Flucindole; Flumezapine; Fluphenazine Decanoate; Fluphenazine Enanthate; Fluphenazine Hydrochloride; Fluspiperone; Fluspirilene; Flutroline; Gevotroline Hydrochloride; Halopemide; Haloperidol; Iloperidone; Imidoline Hydrochloride; Lenperone; Loxapine; Mazapertine Succinate; Mesoridazine; Metiapine; Milenperone; Milipertine; Molindone Hydrochloride; Naranol Hydrochloride; Neflumozide Hydrochloride; Ocaperidone; Olanzapine; Oxiperomide; Penfluridol; Pentiapine Maleate; Perphenazine; Pimozide; Pinoxepin Hydrochloride; Pipamperone; Piperacctazine; Pipotiazine Palmnitate; Piquindone Hydrochloride; Prochlorperazine Edisylate; Prochlorperazine Maleate; Promazine Hydrochloride; Quetiapine; Remoxipride; Quetiapine Remoxipride Hydrochloride; Risperidone; Risperadone Rimcazole Hydrochloride; Seperidol Hydrochloride; Sertindole; Setoperone; Spiperone; Sulpiride; Thioridazine; Thiothixene; Thorazine; Tioperidone Hydrochloride; Tiospirone Hydrochloride; Trifluoperazine Hydrochloride; Trifluperidol; Triflupromazine; Ziprasidone Hydrochloride, analogs, derivative and combinations thereof (see, for example, U.S. Patent Application Nos: 20040019030 and 2002/0123490, the teachings of both of which are hereby incorporated by reference in their entirety).

Exemplary M1 muscarinic receptor antagonists may include telenzepine, trihexyphenidyl (Artane), benztropine (Cogentin), Dicyclomine (Bentyl), biperiden (1-(5-bicyclo[2.2.1]hept-2-enyl)-1-phenyl-3-(1-piperidyl) propan-1-ol), procyclidine (1-cyclohexyl-1-phenyl-3-pyrrolidin-1-yl-propan-1-ol hydrochloride), or scopolamine ((−)-(S)-3-Hydroxy-2-phenyl-propionic acid (1R,2R,4S,7S,9S)-9-methyl-3-oxa-9-aza-tricyclo[3.3.1.02,4]non-7-yl ester).

### Unit Doses and Kits

In another aspect, this disclosure also provides a unit dose comprising a pharmaceutically effective amount of the composition as described herein.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to) treat or prevent a disease or disorder, or to treat the symptoms of the disease or disorder, in a host. Thus, an “effective amount” generally means an amount that provides the desired effect. A “therapeutically effective amount” of a compound with respect to the subject method of treatment refers to an amount of the compound in a preparation which, when administered as part of a desired dosage regimen (to a mammal, e.g., a human), alleviates a symptom, ameliorates a condition, or slows the onset of disease conditions according to clinically acceptable standards for the disorder or condition to be treated, e.g., at a reasonable benefit/risk ratio applicable to any medical treatment.

The actual dosage amount of a composition of this disclosure administered to a subject can be determined by physical and physiological factors such as body weight, severity of condition, the type of disease being treated, previous or concurrent therapeutic interventions, idiopathy of the patient, and on the route of administration. The practitioner responsible for administration will, in any event, determine the concentration of active ingredient(s) in a composition and appropriate dose(s) for the individual subject.

In some embodiments, dosage forms in a unit dose include, for example, tablets, pills, capsules, injections (ampoules), suppositories, etc. In some embodiments, the unit dose is provided in a form of a tablet (e.g., orally disintegrating tablet (ODT)) or a capsule. In some embodiments, the unit dose (e.g., tablet) comprises about 1 mg to 100 mg (e.g., 1 mg, 2 mg, 3 mg, 4 mg, 5 mg, 6 mg, 7 mg. 8 mg, 9 mg, 10 mg, 11 mg, 12 mg, 13 mg, 14 mg, 15 mg, 16 mg, 17 mg, 18 mg, 19 mg, 20 mg. 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg. 37 mg, 38 mg. 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg. 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg. 73 mg. 74 mg. 75 mg, 76 mg, 77 mg, 78 mg. 79 mg, 80 mg, 81 mg, 82 mg, 83 mg, 84 mg, 85 mg, 86 mg, 87 mg, 88 mg, 89 mg, 90 mg, 91 mg, 92 mg. 93 mg. 94 mg, 95 mg, 96 mg, 97 mg, 98 mg. 99 mg, 100 mg) of the baclofen, a stereoisomer thereof, a derivative thereof, an analog thereof, a prodrug thereof, or a pharmaceutically acceptable salt thereof. In some embodiments, the tablet comprises about 50 mg to 300 mg (e.g., about 50 mg, 75 mg, 100 mg, 125 mg, 150 mg, 175 mg. 200 mg. 225 mg, 250 mg. 275 mg, 300 mg) of the composition described herein.

In some embodiments, the unit dose further comprises a foil packaging encapsulating the composition.

In another aspect, this disclosure provides a kit comprising a pharmaceutically acceptable dose unit of the composition described herein.

In some embodiments, in addition to the composition, the kit may include an additional therapeutic agent. In some embodiments, the additional therapeutic agent is selected from a Group II mGluR agonist, Group II mGluR agonist, GSK3β antagonists, NAAG peptidase inhibitors, Group I mGluR antagonist, an antidepressant, an anti-psychotic, an α2-adrenergic agonist, an anticonvulsant, a nicotinic receptor agonist, an endocannabinoid receptor antagonist, a M1 muscarinic receptor antagonist, and an AMPA agonist.

In some embodiments, the kit also includes a container that contains the composition and optionally informational material. The informational material can be descriptive, instructional, marketing, or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In some embodiments, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading.

### Methods of Making Compositions

In yet another aspect, this disclosure further provides a method for preparing a composition or a unit dose described herein. In some embodiments, the method comprises:
(a) passing an active pharmaceutical ingredient through a 80 mesh screen, wherein the active pharmaceutical ingredient comprises baclofen, a stereoisomer thereof, a derivative thereof, an analog thereof, a prodrug thereof, or a pharmaceutically acceptable salt thereof;
(b) blending about 0.1 kg to 0.3 kg (e.g., 0.1, 0.12, 0.14, 0.16, 0.18, 0.2, 0.22, 0.24, 0.26, 0.28, 0.3 kg) of the active pharmaceutical ingredient and about 0.3405 kg to 1.0215 kg (e.g., 0.3405, 0.3905, 0.4405, 0.4905, 0.5405, 0.5905, 0.6405, 0.6905, 0.7405, 0.7905, 0.8405, 0.8905, 0.9405, 0.9905, 1.0215 kg) of the excipient for about 10 minutes at about 25 rpm to obtain a first mixture;
(c) blending about 1.834 kg of the excipient for about 10 minutes at about 25 rpm;
(d) adding to the excipient of step (c) to the first mixture to obtain a second mixture, and passing the second mixture through a 50 mesh screen;
(e) blending the second mixture with about 0.15 kg to about 0.45 kg (e.g., 0.15, 0.17, 0.19, 0.21, 0.23, 0.25, 0.27, 0.29, 0.31, 0.33, 0.35, 0.37, 0.39, 0.41, 0.43, 0.45 kg) of the disintegrating agent, about 0.6 kg to about 0.18 kg (e.g., 0.06, 0.07, 0.08, 0.09, 0.1, 0.11, 0.12, 0.13, 0.14, 0.15, 0.16, 0.17, 0.18 kg) of the sweetener, about 0.009 kg to about 0.027 kg (e.g., 0.009, 0.011, 0.013, 0.015, 0.017, 0.019, 0.021, 0.023, 0.025, 0.027 kg) of the flavoring agent, and about 0.003 kg to about 0.009 kg (e.g., 0.003, 0.0031, 0.0051, 0.0071, 0.009 kg) of the masking agent for about 20 minutes at about 25 rpm to obtain a third mixture; and
(f) passing the lubricating agent through a 30 mesh screen, and blending about 0.6 kg to about 0.18 kg (e.g., 0.06, 0.07, 0.08, 0.09, 0.1, 0.11, 0.12, 0.13, 0.14, 0.15, 0.16, 0.17, 0.18 kg) of the lubricating agent with the third mixture for about 5 minutes at about 25 rpm to obtain the composition.

In some embodiments, the excipient comprises F-MELT. In some embodiments, the disintegrating agent comprises crospovidone. In some embodiments, the lubricating agent comprises sodium stearyl fumarate. In some embodiments, the sweetener comprises sucralose.

In some embodiments, the composition comprises: from 1.197% w/w to 1.463% w/w (e.g., 1.21%. 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46% (w/w)) of R-baclofen, from 80.343% w/w to 98.197% w/w (e.g., 80.84%, 81.34%, 81.84%, 82.34%, 82.84%, 83.34%, 83.84%, 84.34%, 84.84%, 85.34%, 85.84%, 86.34%, 86.84%, 87.34%, 87.84%, 88.34%, 88.84%, 89.34%, 89.84%, 90.34%, 90.84%, 91.34%, 91.84%, 92.34%, 92.84%, 93.34%, 93.84%, 94.34%, 94.84%, 95.34%, 95.84%, 96.34%, 96.84%, 97.34%, 97.84%, 98% (w/w)) of the excipient, from 4.5% w/w to 5.5% w/w (e.g., 4.50%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.50% (w/w)) of the disintegrating agent, from 1.8% w/w to 2.2% w/w (e.g., 1.80%, 1.85%, 1.90%, 1.95%, 2.00%, 2.05%. 2.10%, 2.15%, 2.20% (w/w)) of the lubricating agent, from 1.8% w/w to 2.2% w/w (e.g., 1.80%, 1.85%, 1.90%, 1.95%, 2.00%, 2.05%, 2.10%, 2.15%, 2.20% (w/w)) of the sweetener, from 0.27% w/w to 0.33% w/w (e.g., 0.270%, 0.275%, 0.280%, 0.285%, 0.290%. 0.295%, 0.300%, 0.305%, 0.310%, 0.315%, 0.320%, 0.325%, 0.330% (w/w)) of the flavoring agent, and from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: from 3% w/w to 3.67% w/w (e.g. 3.00%, 3.05%, 3.10%, 3.15%, 3.20%, 3.25%, 3.30%, 3.35%, 3.40%, 3.45%, 3.50%, 3.55%, 3.60%, 3.65% (w/w)) of R-baclofen, from 78.54% w/w to 95.99% w/w (e.g., 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%, 89.04%, 89.54%, 90.04%, 90.54%, 91.04%, 91.54%, 92.04%, 92.54%, 93.04%, 93.54%, 94.04%, 94.54%, 95.04%, 95.54%, 95.99% (w/w)) of the excipient, from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent, from 1.8% w/w to 2.2% w/w (e.g., 1.80%, 1.85%, 1.90%, 1.95%, 2.00%. 2.05%, 2.10%, 2.15%, 2.20% (w/w)) of the lubricating agent, from 1.8% w/w to 2.2% w/w (e.g., 1.80%, 1.85%, 1.90%, 1.95%, 2.00%, 2.05%, 2.10%, 2.15%, 2.20% (w/w)) of the sweetener, from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent, and from 0.09% w/w to 0.11% w/w (e.g., 0.090%, 0.092%, 0.094%, 0.096%, 0.098%, 0.100%. 0.102%, 0.104%. 0.106%, 0.108%, 0.110% (w/w)) of the masking agent.

In some embodiments, the composition comprises: from 6.0% w/w to 7.34% w/w (e.g., 6.0%, 6.1%, 6.2%, 6.3%, 6.4%, 6.5%, 6.6%, 6.7%, 6.8%, 6.9%, 7.0%, 7.1%, 7.2%, 7.3% (w/w)) of R-baclofen, from 75.54% w/w to 92.32% w/w (e.g., 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54%. 89.04%, 89.54%, 90.04%, 90.54%, 91.04%, 91.54%, 92.04% (w/w)) of the excipient, from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent, from 1.8% w/w to 2.2% w/w (e.g., 1.80%, 1.85%, 1.90%, 1.95%, 2.00%, 2.05%. 2.10%. 2.15%, 2.20% (w/w)) of the lubricating agent, from 1.8% w/w to 2.2% w/w (e.g., 1.80%, 1.85%, 1.90%, 1.95%, 2.00%, 2.05%, 2.10%, 2.15%, 2.20% (w/w)) of the sweetener, from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent, and from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

In some embodiments, the composition comprises: from 9% w/w to 11% w/w (e.g., 9%, 9.2%, 9.4%, 9.6%, 9.8%, 10.0%, 10.2%, 10.4%, 10.6%, 10.8%, 11% (w/w)) of R-baclofen, from 72.54% w/w to 88.66% w/w (e.g., 72.54%, 73.04%, 73.54%, 74.04%, 74.54%, 75.04%, 75.54%, 76.04%, 76.54%, 77.04%, 77.54%, 78.04%, 78.54%, 79.04%, 79.54%, 80.04%, 80.54%, 81.04%, 81.54%, 82.04%, 82.54%, 83.04%, 83.54%, 84.04%, 84.54%, 85.04%, 85.54%, 86.04%, 86.54%, 87.04%, 87.54%, 88.04%, 88.54% (w/w)) of the excipient, from 4.5% w/w to 5.5% w/w (e.g., 4.5%, 4.55%, 4.60%, 4.65%, 4.70%, 4.75%, 4.80%, 4.85%, 4.90%, 4.95%, 5.00%, 5.05%, 5.10%, 5.15%, 5.20%, 5.25%, 5.30%, 5.35%, 5.40%, 5.45%, 5.5% (w/w)) of the disintegrating agent, from 1.8% w/w to 2.2% w/w (e.g., 1.80%, 1.85%, 1.90%, 1.95%, 2.00%, 2.05%, 2.10%, 2.15%, 2.20% (w/w)) of the lubricating agent, from 1.8% w/w to 2.2% w/w (e.g., 1.80%, 1.85%, 1.90%, 1.95%, 2.00%, 2.05%, 2.10%, 2.15%, 2.20% (w/w)) of the sweetener, from 0.27% w/w to 0.33% w/w (e.g., 0.270%, 0.275%, 0.280%, 0.285%, 0.290%, 0.295%, 0.300%, 0.305%, 0.310%, 0.315%, 0.320%, 0.325%, 0.330% (w/w)) of the flavoring agent, and from 0.09% w/w to 0.11% w/w (e.g., 0.090%, 0.092%, 0.094%, 0.096%, 0.098%, 0.100%, 0.102%, 0.104%, 0.106%, 0.108%, 0.110% (w/w)) of the masking agent.

### Methods of Use of Compositions

The disclosed compositions can be used for treating or controlling conditions that are associated with autism or fragile X syndrome, for example, Coffin-Lowry syndrome, Cohen syndrome, Duchenne/Becker muscular dystrophies, Neurofibromatosis, Joubert syndrome, Lujan-Fryns syndrome, PTEN mutations, Noonan syndrome, Orstavik syndrome, ARX mutations, CHARGE, Angelman syndrome, Nance-Horan syndrome, Prader-Willi syndrome, Cerebral dysgenesis, or Smith-Lemli-Optiz syndrome. Additional conditions may include pervasive developmental disorders with no identified source and autism and other disorders of brain development. Disorders of brain development may include Autism Spectrum Disorders (Pervasive Developmental Disorders), Rett's syndrome, Childhood Disintegrative Disorder, Asperger syndrome, and Tuberous Sclerosis.

The disclosed compositions can be used for treating or controlling deficits/symptoms, for example, deficits in learning, memory, executive function, attention and/or processing speed. Such deficits can be deficits associated with or observed in subjects with mental retardation, fragile X syndrome, Down's syndrome, and autism; and pervasive developmental disorders, including pervasive developmental disorders with no obvious source.

The disclosed compositions can be used for treating or controlling neuropsychiatric disorders and anxiety disorders, including anxiety disorders that are associated with or observed in subjects that have mental retardation, autism, Down's syndrome, and fragile X syndrome. Such anxiety disorders include, for example, specific phobias, such as phobias of the doctor and dentist; agoraphobia, and separation anxiety. Such disorders can also include, for example, bipolar disorders, repetitive and stereotyped behavior, obsessive and compulsive traits/disorders, aggressive behavior, schizophrenia, hyperactivity, pain, itching, sensory hyperarousal, seizures, behavioral problems, sleep disorders (including insomnia, hypersomnia and abnormal behaviors during sleep).

The disclosed compositions can also be used for treating or controlling gastrointestinal disorders and metabolic disorders in subjects with mental retardation, fragile X syndrome, Down's syndrome, and autism. Autistic behavior (deficits in social interaction, verbal and non-verbal communication, and restricted/repetitive behaviors or interests) in subjects with autism, mental retardation, fragile X syndrome, and Down's syndrome can also be treated by the methods of the invention.

Accordingly, this disclosure also provides a method for treating a condition in a subject in need thereof by administering to the subject a therapeutically effective amount of the composition disclosed herein.

Subjects treated by the methods described herein can have at least one condition selected from the group consisting of a sensory hyperarousal disorder, an anxiety disorder, a seizure disorder, a gastrointestinal disorder, a sleep disorder, aggressive or aberrant behavior, and an impaired cognitive function. Subjects treated by the methods described herein can also have at least one condition selected from a social interaction abnormality, limited interests and repertoire of behaviors, and a social avoidance condition. Subjects (e.g., humans, also referred to herein as “patients”) treated by the methods of the invention can have a cognitive impairment, such as an impairment in reaction time, eye tracking, motor coordination, gait, oral-motor function, communication, learning, attention, executive function, reaction time, learning, information processing, conceptualization, problem-solving, verbal fluency or memory (e.g., memory consolidation, short-term memory, working memory, long-term memory, declarative memory or procedural memory).

Impairment in a cognitive function treated by the methods described herein can be an impairment in attention, which is the capacity or process of selecting out of the totality of available sensory or affective stimuli, those stimuli that are most appropriate or desirable for focus at a given time (Kinchla, R. A., et al., Annu. Rev. Psychol. 43:711-742 (1992)). The impairment in a cognitive process can be an impairment in executive function, which are neuropsychological functions such as decision-making, planning, initiative, assigning priority, sequencing, motor control, emotional regulation, inhibition, problem-solving, planning, impulse control, establishing goals, monitoring results of action and self-correcting (Elliott, R., Br. Med. Bull. 65:49-59 (2003)). The cognitive impairment can be an impairment in alertness, wakefulness, arousal, vigilance, and reaction time information processing, conceptualization, problem-solving and/or verbal fluency. One of skill in the art would be capable of identifying and evaluating the impairment in a cognitive function in the individual.

An “effective amount,” also referred to herein as a “therapeutically effective amount,” when referring to the amount of a compound or composition (e.g., baclofen, a M1 muscarinic receptor antagonist) is defined as that amount, or dose, of a compound or composition that, when administered to a subject, is sufficient for therapeutic efficacy (e.g., an amount sufficient decrease to exhibit a clinical improvement in a behavior or mental cognitive test score; alleviate sensory hyperarousal disorder, an anxiety disorder, a seizure disorder, a gastrointestinal disorder, a sleep disorder, prevent weight gain, decrease obsessive compulsive tendencies and manners).

The methods can be accomplished by the administration of the composition by enteral means. The route of administration can be by oral ingestion (e.g., tablet, capsule form) of the composition.

In some embodiments, the composition can be administered in a dose of between about 0.01 mg/kg to about 0.1 mg/kg; about 0.001 mg/kg to about 0.01 mg/kg; about 0.001 to about 0.05 mg/kg; about 0.1 mg/kg to about 1 mg/kg body weight; about 1 mg/kg to about 5 mg/kg body weight; or between about 5 mg/kg to about 15 mg/kg body weight.

In some embodiments, the composition can be administered in doses of about 0.1 mg, about 1 mg, about 2 mg, about 2.5 mg, about 5 mg, about 10 mg, about 15 mg, about 20 mg, about 25 mg, about 30 mg, about 40 mg, about 45 mg, about 50 mg, about 60 mg, about 80 mg, 100 mg, about 200 mg, about 250 mg, about 300 mg, about 350 mg, about 400 mg, about 500 mg, about 600 mg, about 700 mg, about 900 mg, about 1000 mg, about 1200 mg, about 1400 mg, about 1600 mg or about 2000 mg, or any combination thereof. The composition can be administered once a day or multiple (e.g., two, three, four, five) times per day.

In some embodiments, the composition can be administered at a dosing regimen that includes progressive or escalating increases in the composition over time of treatment. For example, a subject can be treated with baclofen (e.g., R-baclofen, such as between about 20 to about 40 mg per day) at a dose of about 2 mg/day at days 1, 2, 3 of treatment; about 4 mg/day at days 4, 5, 6 of treatment; about 6 mg/day at days 7, 8, 9 of treatment; about 20 mg/day at days 13, 14, 15 of treatment and about 30 mg/day at days 16, 17 and 18 of treatment.

The composition can be administered to the human in a selected dose (e.g., about 10 mg dose taken 3 times a day or about 15 mg dose given as three doses each of which is about 5 mg) while monitoring improvements in the human (e.g., cognition, behavior). If the human does not exhibit any improvement, the compositions employed in the methods can be increased, decreased or stopped until a beneficial effect is observed. For example, if treatment began with three (3) doses of about 10 mg daily and the human subsequently exhibited no apparent improvement, the dose could be increased to three (3) doses of about 15 mg a day, decreased to two (2) doses of about 10 mg a day or treatment could be halted for a single dose, a number of days or weeks and subsequently commenced following the “mini-drug holiday.” “Mini-drug holiday.” as used herein, refers to removal of the human from treatment or a decrease in the dose of the compound, followed by re-introduction of the treatment, at a dose equivalent to, below or in excess of the dose the human received prior to the mini-drug holiday.

The dosage and frequency (single or multiple doses) administered to an individual can vary depending upon a variety of factors, including the duration of condition of the subject (e.g., sensory hyperarousal disorder, anxiety disorder, seizure disorder, gastrointestinal disorder, sleep disorder, an impaired cognitive function, weight gain, obsessive compulsive behaviors); the route of administration of the compound; size, age, sex, health, body weight, body mass index, and diet of the recipient; nature and extent of symptoms of the disorder being treated (e.g., sensory hyperarousal disorder, anxiety disorder, seizure disorder, gastrointestinal disorder, sleep disorder, impaired cognitive function), kind of concurrent treatment (e.g., behavioral modification, anti-depressant medications, α2-adrenergic agonists, anticonvulsants, a nicotinic receptor agonist, an endocannabinoid receptor antagonist, AMPA agonists, anti-psychotics), complications from, for example, a sensory hyperarousal disorder, anxiety disorder, seizure disorder, gastrointestinal disorder, sleep disorder or impaired cognitive function; or other health-related problems. Other therapeutic regimens or agents can be used in conjunction with the methods of the present invention. For example, the administration of the compounds employed in the methods of the invention can be accompanied by behavioral modifications, anti-depressant medications, and antipsychotic medications. Adjustment and manipulation of established dosages (e.g., frequency and duration) are well within the ability of those skilled in the art.

In some embodiments, the composition can be administered alone or can be co-administered to the patient. Co-administration may include simultaneous or sequential administration of the disclosed composition and one or more additional therapeutic agents (e.g., a M1 muscarinic receptor antagonist). The mode of administration can be conducted sufficiently close in time to each other so that the effects on the subject are maximal.

In some embodiments, the composition can be administered to a subject with (e.g., before, concomitantly, sequentially, or after) administration of one or more additional therapeutic agents that are employed to treat a particular disorder or condition in the subject. For example, the compostion can be administered with at least one member selected from a Group II mGluR agonist, Group II mGluR agonist, GSK3β antagonists, NAAG peptidase inhibitors, Group I mGluR antagonist, an antidepressant, an anti-psychotic, an α2-adrenergic agonist, an anticonvulsant, a 30 nicotinic receptor agonist, an endocannabinoid receptor antagonist, a M1 muscarinic receptor antagonist, and an AMPA agonist.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “formulation,” in general, refers to a preparation that includes at least one pharmaceutically active compound optionally in combination with one or more excipients or other pharmaceutical additives for administration to a subject. In general, particular excipients and/or other pharmaceutical additives are typically selected with the aim of enabling a desired stability, release, distribution, and activity of active compound(s) for applications.

In general, pharmaceutically acceptable salts include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, carbonate, ascorbate, succinate, maleate, gentisinate, fumarate, gluconate, glucuronate, saccharate, formate, carboxylate, benzoate, glutamate, sulfonate, methanesulfonate, ethanesulfonate, benzensulfonate, p-toluenesulfonate, selenate, and pamoate (i.e., 1,1′-methylene-bis-(2-hydroxy-3-naphthoate)) salts of compounds.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British pharmacopeia (BP), National Formulary (NF), European Pharmacopoeia (EP), Japanese Pharmacopeia (JP), Chinese Pharmacopoeia (ChP) etc.). Pharmaceutical grade further incorporates suitability for administration by means including topical, ocular, parenteral, nasal, pulmonary tract, mucosal, vaginal, rectal, intravenous, and the like.

Doses are often expressed in relation to body weight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

It is noted here that, as used in this specification and the appended claims, the singular forms “a.” “an,” and “the” include plural reference unless the context clearly dictates otherwise.

The terms “including.” “comprising.” “containing.” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Examples are provided below to further demonstrate the important aspects of the present disclosure, but it should be understood that the disclosure is not limited thereto.

### Example 1

### R-Baclofen Orally Disintegrating Formulations and Characterization

### Materials and Reagents

The materials and reagents used during formulation development and analysis of the R-baclofen orally disintegrating tablets (ODTs) are listed in Table 1.

TABLE 1
Materials and Reagents

| Material/Reagent | Supplier |
|---|---|
| R-baclofen API | Seaside |
| Purified Water (H₂O) | In-House Milli Q |
| Potassium Phosphate, Monobasic (KH₂PO4) | JT Baker* |
| Phosphoric Acid, 85% (H₃PO₄) | JT Baker* |
| Acetonitrile (ACN) | JT Baker* |
| Food Coloring/Dye | Various |
| Anhydrous Methanol (for Karl Fischer) | EMD |
| Water Standard, 0.1% (for Karl Fischer) | EMD |
| ProSolv HD90 (Silicified MCC) | JRS Pharma |
| ProSolv SMCC50 (Silicified MCC) | JRS Pharma |
| Pharmaburst 500 | SPI Pharma |
| Lubripharm (Sodium Stearyl Fumarate) | SPIPharma |
| Sucralose | JK Sweet |
| Sucrose | Spectrum |
| Lactose | Foremost |
| N-C Strawberry Flavor | FONA |
| N-C Orange Flavor | FONA |
| Masking Agent | FONA |
| F-MELT Type C | Fuji Health Science |
| Polyplasdone XL (Crospovidone) | ISP |
| Pruv (Sodium Stearyl Fumarate) | JRS Pharma |
| Pearlitol 100SD (Mannitol) | Roquette |
| Pearlitol 300DC (Mannitol) | Roquette |
| RXcipients FM1000 (Calcium Silicate) | Mutchler/Huber |
| Cabosil (Colloidal Silicon Dioxide) | Cabot |
| L-HPC (Low-Substituted Hydroxypropyl | Shin Etsu |
| Cellulose) | |
| Ludiflash (see Table X) | BASF |
| PanExcea ODT (see Table X) | Mallinckrodt |

### Equipment and Supplies

The equipment and supplies listed in Table 2 were used during R-baclofen ODT formulation development and testing.

TABLE 2
Equipment and Supplies

| Equipment/Supply | Supplier |
|---|---|
| Friability Tester | Varian |
| Hardness Tester | Varian |
| Rotary Tablet Press (Mini Press) | Globe Pharma |
| Single Station Tablet Press | Globe Pharma |
| Turbula Blender | Glen Mills, Inc. |
| Karl Fischer Titrator | EMD Aquastar AQC34 |
| Analytical Balance | Mettler-Toledo |
| Top-Loading Balance | Mettler-Toledo |
| Thermometer | VWR |
| Timer | VWR |
| Disintegration Apparatus | Hanson |
| Spatulas | VWR |
| 0.45 μm Nylon Syringe Filters | Pall Gelman |
| Volumetric Pipettes | VWR |
| Adjustable Pipettes | Various |
| Transfer Pipettes | VWR |
| Volumetric Flasks | VWR |
| Glass Beakers | VWR |
| Graduated Cylinders | VWR |
| Glass Solvent Bottles | VWR |
| 60 cc Round White HDPE | Drug Plastics and |
| Bottles | Glass Co. |
| 28 mm Round HDPE Closures | Drug Plastics and |
| | Glass Co. |
| Desiccant Canister | Sorbit |
| HPLC Vials w/Caps | Agilent |
| Di!: tital Calipers | Fisher Scientific |
| Induction Sealer | Induction Sealer |
| Blister Packing Machine | InPack |
| Thermo-Forms | Klockner Pentaplast |
| Thermo-Form Backing | Constantia Huek Foils |

### Controlled Environment Equipment

The equipment detailed in Table 3 were used to provide controlled environments for the R-baclofen ODT prototypes.

TABLE 3
Controlled Environment Equipment

| Equipment Type | Condition | Manufacturer |
|---|---|---|
| Stability Chamber | 25 ± 2° C./60 ± 5% RH | Lunaire |
| Stability Chamber | 40 ± 2° C./75 ± 5% RH | Lunaire |

### Analytical Equipment

The HPLC systems used to analyze samples were Waters 2695 Separations Modules with 2487 Dual Wavelength Detectors.

### Test Methods

### Method Parameters

The procedures used for content uniformity, potency, purity, water content, and physical testing (hardness, friability, disintegration, simulated wetting test (SWT)) for R-baclofen ODT prototypes are provided below.
1. 2 mg ODT assay sample preparation

Samples were prepared by adding 3 tablets to a 25-mL volumetric flask, producing a nominal concentration of 240 μg/mL.
2. 10 mg ODT assay sample preparation

Samples were prepared by adding 3 tablets to a 100-mL volumetric flask, producing a nominal concentration of 300 μg/mL.
3. Karl Fischer water content samples were prepared using 3 tablets.

### In Vivo Disintegration Testing

For in vivo disintegration testing, participants were instructed to place the placebo ODT on their tongue and apply gentle pressure to the ODT against the roof of their mouth. They could move the tablet gently against the roof of their mouth and swallow, but could not tumble or chew the tablet. A timer was started once the ODT was placed in the mouth, and participants were instructed to stop the timer once the tablet completely disintegrated and the tablet structure could no longer be detected.

### Odt Formulation Development

Initial R-baclofen ODT formulation development focused on evaluating a variety of excipients used to prepare placebo formulations. Blends were prepared, compressed into tablets, and evaluated on hardness, simulated wetting test (SWT), in vivo disintegration time, and friability. The main criterion for a successful ODT was an in vivo disintegration time of approximately 15 seconds or less. This time was identified as necessary for patient compliance in a pediatric cohort.

Tablet hardness was monitored and adjusted to provide tablets with enough physical strength to minimize friability and withstand typical handling conditions during manufacturing and packaging, but also to allow tablets to disintegrate quickly when in contact with moisture.

Because in vivo disintegration times could vary among individual volunteers, an additional in vitro test was developed as an independent assessment to predict how well an ODT may disintegrate in the mouth. The simulated wetting test (SWT) was used successfully to compare formulations and differences in SWT correlated well with differences in in vivo disintegration times.

Once tablets with acceptable physical characteristics were identified, a sweetener, flavorants, a masking agent, and R-baclofen API were incorporated into the formulations. Formulations were assessed subjectively for taste and mouth feel, and were also analyzed for blend uniformity and content uniformity at the 2 mg and 10 mg dosage strengths. Once acceptable formulations were identified, prototype ODTs were produced, packaged, and monitored for stability at 25° C./60% RH and 40° C./75% RH for six months.

### Small Scale Placebo Development (Formulations 1-4)

Initially, 20 g batches of four formulations were prepared and used to produce tablets at a small scale on a single-station manual tablet punch. The formulations contained 93% (w/w) filler/binder, 5% (w/w) Polyplasdone XL (crospovidone), and 2% (w/w) Pruv (sodium stearyl fumarate). The four fillers were mannitol (Pearlitol 300DC), sucrose (crystal), SMCC (ProSolv HD90), and lactose (fast-flow). The four formulations are detailed in Table 4.

TABLE 4
Placebo Formulations 1-4

| | Formulation | | |
|---|---|---|---|
| | Filler/Binder | | Lubricant |
| Formulation | (93% of | Disintegrant | (2% of |
| Number | Formulation) | (5% of Formulation) | Formulation) |
| 1 | Mannitol | Polyplasdone XL | Pruv |
| 2 | Sucrose | Polyplasdone XL | Pruv |
| 3 | SMCC | Polyplasdone XL | Pruv |
| 4 | Lactose | Polyplasdone XL | Pruv |

The powders were sieved through a 600 μm screen prior to weighing, and each formulation was blended on a Turbula Blender at 32 RPM for 10 minutes. Tablets were pressed using 9/32 inch round, flat tooling with the top score, targeting a tablet weight of 100 mg. Tablets were compressed with 1000 to 4000 PSI of force on a single-station manual tablet press to assess each formulation's capacity for tablet hardness. The hardness results are shown in Table 5.

TABLE 5
Tablet Hardness Produced from Formulations 1-4

| Formulation | Hardness Range (kp) |
|---|---|
| 1 (mannitol) | 1.0-3.1 |
| 2 (sucrose) | 0 |
| 3 (SMCC) | 10.4-14.5 |
| 4 (lactose) | 2.6-5.3 |

The initial assessment of Formulations 1-4 showed that the grade of sucrose used was not compressible and not suitable for tablet production. Formulations 1, 3, and 4 produced tablets with a smooth, shiny appearance that did not show signs of excessive friability when rolled between the fingers. Additional tablets of Formulation 1 and 3 were pressed at 1000 and 3000 PSI and tested for SWT and in vivo disintegration time. The results are shown in Table 6.

TABLE 6
Tablet Hardness Produced from Formulations 1-4

| | Compression | | In Vivo |
|---|---|---|---|
| | Force | SWT | Disintegration |
| Formulation | <PSD | (sec) | (sec) |
| 1 (mannitol) | 1000 | 19.6-22.0 | 13.4-19.1 |
| 1 (mannitol) | 3000 | 32.0-45.8 | 19.6-27.8 |
| 3 (SMCC) | 1000 | 67-148 | 65-73 |
| 3 (SMCC) | 3000 | 233-283 | 146-183 |

Tablets prepared using mannitol as the filler showed good disintegration times for an ODT formulation. Additionally, mannitol tablets exhibited a sweet taste and cool, smooth mouth feel. SMCC tablets were much slower to disintegrate, had a dust-like taste, and were slightly gritty due to the insoluble SMCC. An increase in compression force significantly increased the times obtained for SWT and in vivo disintegration with both mannitol and SMCC.

### Large Scale Placebo Development (Formulations 5-16)

After the initial assessment on the single station tablet punch, additional formulations were prepared at a larger scale (200-300 gram batch size) and ODTs were produced using a rotary tablet press. Placebo Formulations 5-16 are shown in Table 7. These formulations were prepared by combining the components in a glass blending vessel and blending for 5 minutes at 32 RPM on a Turbula blender. The blends were then sieved through a 600 μm screen and blended an additional 5 minutes on the Turbula blender. Tablets were pressed using the same 9/32 inch round, flat tooling with top score and with a target weight of 100 mg.

TABLE 7
Placebo Formulations 5-16

| | Amount of Component (% w/w) in Each Formulation | | | | | | | | | | | |
|---|---|---|---|---|---|---|---|---|---|---|---|---|
| Component | 5 | 6 | 7 | 8 | 9 | 10 | 11 | 12 | 13 | 14 | 15 | 16 |
| Pearlitol 300DC | 87.5 | 77.5 | 77.5 | | 57.5 | | | | | | | 37.5 |
| Pearlitol 100SD | | | | | | | | 57.5 | 52.5 | | | |
| ProSolv HD90 | | | | | | 57.5 | | | 15.0 | | | 20.0 |
| Ludiflash | | | | 97.5 | | | | | | | | |
| PanExcea ODT | | | | | | | 82.5 | | | | | |
| Phannaburst 500 | | | | | | | | | | 98.0 | | |
| F-MELT, Type C | | | | | | | | | | | 93.0 | |
| Polyplasdone XL | 10.0 | 20.0 | | | 20.0 | 20.0 | 15.0 | 20.0 | 15.0 | | 5.0 | 20.0 |
| L-HPC | | | 20.0 | | | | | | | | | |
| RXcipients | | | | | 20.0 | 20.0 | | 20.0 | 15.0 | | | 20.0 |
| FM1000 | | | | | | | | | | | | |
| Lubriphann | | | | | | | | | | 2.0 | | |
| Pruv | 2.0 | 2.0 | 2.0 | 2.0 | 2.0 | 2.0 | 2.0 | 2.0 | 2.0 | | 2.0 | 2.0 |
| Cabosil | 0.5 | 0.5 | 0.5 | 0.5 | 0.5 | 0.5 | 0.5 | 0.5 | 0.5 | | | 0.5 |

Formulations 5, 6, and 7 were prepared first and analyzed for hardness, friability, SWT, and in vivo disintegration time, and the results are shown in Table 8.

TABLE 8
Testing Results for Formulations 5-7

| | | | | In vivo |
|---|---|---|---|---|
| Formulation | Hardness | Friability | SWT | Disintegration |
| Number | [symbol] Oro) | (% weight lost) | (sec) | (sec) |
| 5 | 2.2-2.4 | 0.08% | 28.8-32.2 | 17-25 |
| 6* | 3.2-3.9 | 0% | 45.4-48.3 | 18-29 |
| 6b* | 2.2-2.9 | NT | 21.6-25.3 | 13-20 |
| 7* | 2.4-3.2 | 0.13% | 56.2-56.6 | 26-35 |
| 7b* | 2.8-4.9 | NT | 148-155 | 62-79 |

[symbol]

indicates data missing or illegible when filed

Formulations 5-7 used mannitol (Pearlitol 300DC) as the filler, which produced tablets with sufficient hardness to minimize friability, while maintaining relatively rapid SWT and in vivo disintegration times compared to MCC (as seen in Formulation 3, above). These formulations also imparted a cool taste and smooth mouth feel. Formulations 5 and 6 compared two levels of disintegrant (10% vs. 20% Polyplasdone XL), while Formulations 6 and 7 compared two different types of disintegrant (Polyplasdone XL vs. L-HPC). Formulations 6 and 7 were also used to produce tablets using two different compression forces, resulting in two tablet hardness ranges.

The effect of compression force, and subsequent tablet hardness, was evident when comparing Formulation 6 with 6b and Formulation 7 with 7b. Small changes in tablet hardness (approximately 1 kp) affected disintegration, where softer tablets disintegrated much more rapidly than harder tablets. Doubling the amount of Polyplasdone XL (disintegrant) in the formulation from 10% to 20% (compare Formulation 5 to Formulation 6b to control for tablet hardness) also decreased SWT and in vivo disintegration time. Substituting L-HPC for Polyplasdone XL as the formulation disintegrant worsened the perfomance of the ODT (increased SWT and in vivo disintegration time).

Formulations 5-7 showed that tablet hardness in the 2-3 kp range enhanced ODT performance compared to harder tablets, but were also durable enough to minimize friability problems. Increasing the content of Polyplasdone XL in the formulation also enhanced ODT performance.

Formulations 8-16 assessed additional excipients designed specifically for direct-compression ODT applications. These “designer” excipients are described in Table 9.

TABLE 9
Designer Excipients Evaluated During Formulation Development

| Excipient | Type | Description |
|---|---|---|
| Pearlitol 100SD | Filler/Binder | Granulated mannitol of smaller particle size (compared |
| | | to Pearlitol 300DC) |
| Ludiflash | Filler/Binder/ | Co-processed excipient containing 90% mannitol, 5% |
| | Disintegrant | crospovidone, and 5% polyvinyl acetate |
| PanExcea ODT | Filler/Binder/ | Co-processed excipient containing 75% of a |
| | Dispersion Aid | polyhydric sugar alcohol and 25% of a silicate salt |
| Pharmaburst 500 | Filler/Binder/ | Co-processed excipient containing 73-93% polyols plus |
| | Disintegrant | disintegrant |
| F-MELT, Type C | Filler/Binder/ | Co-processed excipient containing mannitol, xylitol, |
| | Disintegrant/ | MCC, crospovidone, and anhydrous dibasic calcium |
| | Dispersion Aid | phosphate |
| RXcipients | Disintegrant | Calcium silicate (wicking action aids dispersion) |
| FM1000 | | |
| Lubripharm | Lubricant | Sodium stearyl fumarate |

Multiple tablet batches of Formulations 8-16 were prepared to evaluate different tablet hardness ranges and their subsequent effects on friability, SWT, and in vivo disintegration. Results for “Low” and “High” tablet hardness for each formulation are shown in Table 10.

TABLE 10
Testing Results for Formulations 8-16

| | | | | In vivo |
|---|---|---|---|---|
| Formulation | Hardness | Friability | SWT | Disintegration |
| Number* | (kp) | (% weight lost) | (sec) | (sec) |
| 8-low | 2.4-3.4 | NT | 17.6-21.2 | NT |
| 8-high | 3.0-3.4 | 0.09% | 35.8-39.8 | NT |
| 9-low | 1.8-3.0 | 0.09% | 15.2-17.6 | 10-14 |
| 9-high | 2.4-4.0 | NT | 18.5-19.6 | NT |
| 10-low | 3.4-3.8 | 0% | 18.5-23.8** | 8-10 |
| 10-high | 4.2-8.2 | NT | 12.0-14.8 | NT |
| 11-low¹ | 2.2-3.2 | 0.09% | 16.5-20.7 | 11-15 |
| 12-low¹ | 2.4-2.7 | 0% | 16.1-21.3 | 12-21 |
| 13-low | 1.9-2.5 | 0.05% | 16.9-34.9* | 11-17 |
| 13-high | 2.6-3.0 | NT | 12.2-15.1 | 14-16 |
| 14-low | 2.0-2.1 | 0% | 19.4-24.8 | 11-23 |
| 14-high | 3.3-3.6 | 0% | 23.6-26.4 | 15-18 |
| 15-low | 1.5-1.9 | 0% | 10.6-19.8** | 8-17 |
| 15-high | 2.7-3.5 | NT | 26.7-33.6 | 18-23 |
| 16-low | 1.5-1.9 | 0.11% | 15.7-22.4** | 8-20 |
| 16-high | 3.0-4.1 | NT | 13.5-16.0* | 10-13 |

*Formulations used to compare ODTs prepared m “low” and “high” hardness ranges
**Tablets split quickly, preventing complete wetting during testing. Times may be skewed high.
¹Only one hardness range was evaluated NT = Not Tested

The data shown in Table 10 were used to assess the effects of using different formulation compositions or components on ODT performance. The following main comparisons were made to assess which formulation could provide sufficient tablet strength (low friability) and the fastest SWT and in vivo disintegration times, as well as impart an acceptable taste and mouth feel:
1. The particle size of mannitol used in the formulation (Formulation 9 vs. Formulation 12). Formulations 9 and 12 used Pearlitol 300DC (approximately 300 μm particle size) and Pearlitol 100SD (approximately 100 μm particle size), respectively.
2. The effectiveness of calcium silicate (RXcipients FM1000) to disintegrate ODTs rapidly when used in conjunction with mannitol or SMCC (Formulation 9 vs. Formulation 10). Formulation 9 used Pearlitol 300DC while Formulation 10 used ProSolv HD90. Both formulations contained 20% calcium silicate.
3. The effect of using some SMCC in conjunction with calcium silicate in a mostly mannitol formulation (Formulation 12 vs. Formulation 13). Formulation 12 contained mannitol and calcium silicate. Formulation 13 reduced the amount of mannitol, crospovidone (Polyplasdone XL), and calcium silicate in the formulation, but included some SMCC.
4. The effect of using a larger particle size mannitol (100 μm vs. 300 μm) and also increasing the amount of SMCC, crospovidone, and calcium silicate in the formulation (Formulation 13 vs. Formulation 16). Formulation 13 used 100 μm particle size mannitol and 15% each of SMCC, crospovidone, and calcium silicate, while Formulation 16 used 300 μm particle size mannitol and 20% each of SMCC, crospovidone, and calcium silicate.
5. The effectiveness of four ODT excipients used as the main component of the formulation (Formulations 8, 11, 14, and 15).

Friability was acceptable for all tablet formulations, and tablets with greater hardness tended to produce slower SWT and in vivo disintegration times. In general, a tablet hardness of 1-3 kp provided adequate tablet strength while allowing acceptable in vivo disintegration times. Comparison 1 showed that 300 μm particle size mannitol (Pearlitol 300DC) produced tablets with slightly quicker SWT and in vivo disintegration times than 100 μm particle size mannitol (Pearlitol 100SD). Comparison 2 showed that tablets with calcium silicate had faster disintegration times when combined with SMCC compared to mannitol.

Comparison 3 showed that SWT and in vivo disintegration time could be shortened by including some SMCC with calcium silicate in a mannitol formulation. Comparison 4 showed that SWT and in vivo disintegration could be improved further by increasing SMCC, crospovidone, and calcium silicate components from 15% to 20% of the formulation, as well as using 300 μm particle size mannitol instead of 100 μm particle size mannitol.

The ODT excipient Formulations 8 (Ludiflash), 11 (PanExcea ODT), 14 (Pharmaburst 500), and 15 (F-MELT, Type C) produced tablets with minimal friability and quick disintegration times. Formulation 15 (F-MELT. Type C) produced ODTs with the quickest SWT and in vivo disintegration times, as compared to the other tested ODT excipients.

### Flavor and Taste-Masking Development

Once potential formulation candidates were identified, evaluation was performed to determine the levels of flavorant, sweetener, and API taste-masking agent that should be used in the ODT formulation. Additional batches of Formulations 9, 15, and 16 (all placebo) were prepared using masking agent, sucralose, orange flavor, and strawberry flavor. These formulations are listed in Table 11.

TABLE 11
Placebo Formulations for Flavor Comparison

| Formulation | Composition |
|---|---|
| 9 (plus orange) | contained 0.3% orange flavor and 0.1% masking agent |
| | (reduced Pearlitol 300DC by 0.4%) |
| 9 (plus orange and sucralose) | contained 2% sucralose, 0.3% orange flavor, and 0.1% |
| | masking agent (reduced Pearlitol 300DC by 2.4%) |
| 15 (plus strawberry) | contained 0.3% strawberry flavor and 0.1% masking agent |
| | (reduced F-MELT by 0.4%) |
| 15 (plus strawberry and sucralose) | contained 2% sucralose, 0.3% strawberry flavor and 0.1% |
| | masking agent (reduced F-MELT by 2.4%) |
| 16 (plus strawberry) | contained 0.3% strawberry flavor and 0.1% masking agent |
| | (reduced Pearlitol 300DC by 0.4%) |
| 16 (plus strawberry and sucralose) | contained 2% sucralose, 0.3% strawberry flavor and 0.1% |
| | masking agent (reduced Pearlitol 300DC by 2.4%) |

The formulations listed in Table 12 were evaluated for taste and mouth feel. Overall, Formulation 15 was the most pleasant, and strawberry flavor with sucralose (sweetener) present was preferred. It was determined that strawberry flavor with sucralose produced a better combination of taste and sweetness. It was decided that a formulation containing 0.1% masking agent, 0.3% strawberry flavor, and 2% sucralose would be evaluated for taste with R-baclofen API present. The formulation in Table 12 was used to prepare 10 mg R-baclofen ODTs (150 mg tablet weight), which were used for further taste evaluation. It was concluded that the formulation provided enough sweetness and strawberry flavor, and sufficiently masked the taste of the R-baclofen APL

TABLE 12
Formulation 15 (10 mg R-baclofen ODT,
150 mg Total Weight)

| Component | % Formulation |
|---|---|
| F-MELT, Type C | 83.93 |
| R-baclofen API | 6.67 |
| Polyplasdone XL | 5.00 |
| Sucralose | 2.00 |
| Pruv | 2.00 |
| Strawberry Flavor | 0.30 |
| Masking Agent | 0.10 |

### Active R-Baclofen ODT Formulation Development

Three of the above placebo formulations were selected for subsequent formulation development with R-baclofen APL Formulation 15 was selected as the lead formulation due to superior physical attributes (quick disintegration, low friability, and good taste and mouth feel) and because F-MELT, Type C was a more established “designer” ODT excipient (available in the United States since 2006). Formulations 14 and 16 were selected as alternative formulations.

Formulation 14 (Pharmaburst 500) was selected because it also produced ODTs with acceptable SWT, in vivo disintegration, taste, and mouth feel. Formulation 16 was selected because it did not rely on a premixed ODT excipient for the bulk of the formulation, and it also produced tablets with very fast SWT and in vivo disintegration times. Formulation 16 was least desirable, however, because it had a grittier mouth feel due to the presence of significant amounts of SMCC in the formulation. The final compositions of the lead formulation and the two alternates for both 2 mg and 10 mg tablets are shown in Tables 13, 14, and 15.

TABLE 13
Lead-Formulation 15 (F-MELT, Type C)

| | 2 mg R-baclofen ODT | | 10 mg R-baclofen ODT | |
|---|---|---|---|---|
| | | Weight/ | | Weight/ |
| | % | Tablet | % | Tablet |
| Component | Formulation | (nm) | Formulation | (nm) |
| F-MELT | 89.27 | 133.90 | 83.93 | 125.90 |
| R-baclofen | 1.33 | 2.00 | 6.67 | 10.00 |
| Polyplasdone XL | 5.00 | 7.50 | 5.00 | 7.50 |
| Sucralose | 2.00 | 3.00 | 2.00 | 3.00 |
| Pruv | 2.00 | 3.00 | 2.00 | 3.00 |
| Strawberry Flavor | 0.30 | 0.45 | 0.30 | 0.45 |
| Masking Agent | 0.10 | 0.15 | 0.10 | 0.15 |
| Total | 100.00 | 150.0 | 100.00 | 150.00 |

TABLE 14
Alternate 1-Formulation 14 (Pharmaburst 500)

| | 2 mg R-baclofen | | 10 mg R-baclofen | |
|---|---|---|---|---|
| | | Weight/ | | Weight/ |
| | % | Tablet | % | Tablet |
| Component | Formulation | (mg) | Formulation | (mg [symbol] |
| Pharmaburst | 94.27 | 141.40 | 88.93 | 133.40 |
| R-baclofen | 1.33 | 2.00 | 6.67 | 10.00 |
| Lubrioharm | 2.00 | 3.00 | 2.00 | 3.00 |
| Sucralose | 2.00 | 3.00 | 2.00 | 3.00 |
| Strawberry Flavor | 0.30 | 0.45 | 0.30 | 0.45 |
| Masking Agent | 0.10 | 0.15 | 0.10 | 0.15 |
| Total | 100.00 | 150.0 | 100.00 | 150.0 |

[symbol]

indicates data missing or illegible when filed

TABLE 15
Alternate 2-Formulation 16 (SMCC/Mannitol)

| | 2 mg R-baclofen | | 10 mg R-baclofen | |
|---|---|---|---|---|
| | | Weight/ | | Weight/ |
| | % | Tablet | % | Tablet |
| Component | Formulation | (mg) | Formulation | (mg) |
| Pearlitol 300DC | 33.77 | 50.65 | 28.43 | 42.65 |
| ProSolv SMCC50 | 20.00 | 30.00 | 20.00 | 30.00 |
| RXcipients FM1000 | 20.00 | 30.00 | 20.00 | 30.00 |
| Polyplasdone XL | 20.00 | 30.00 | 20.00 | 30.00 |
| R-baclofen | 1.33 | 2.00 | 6.67 | 10.00 |
| Sucralose | 2.00 | 3.00 | 2.00 | 3.00 |
| Pruv | 2.00 | 3.00 | 2.00 | 3.00 |
| Cabosil | 0.50 | 0.75 | 0.50 | 0.75 |
| Strawberry Flavor | 0.30 | 0.45 | 0.30 | 0.45 |
| Masking Agent | 0.10 | 0.15 | 0.10 | 0.15 |
| Total | 100.00 | 150.00 | 100.00 | 150.00 |

The Lead Formulation and Alternates 1 and 2 were prepared and evaluated for blend uniformity and content uniformity to determine if standard blending and tableting procedures were compatible with the 2 mg and 10 mg R-baclofen dosage strengths. These ODT prototypes were also packaged and evaluated for stability at 25° C./60% RH and 40° C./75% RH.

Blends (110 to 150 grams) were prepared by combining formulation components in a glass blending vessel with the R-baclofen API sandwiched between layers of excipient in the middle of the vessel. The formulations were blended for 5 minutes at 32 RPM on a Turbula blender, sieved through a 600 μm screen, and blended for an additional 5 minutes.

Blend uniformity of each batch was tested prior to tablet production. The blended formulations were tested for uniformity by pouring the blend in a line onto aluminum foil and obtaining one sample from the left, middle, and right areas of the blend. The uniformity results for each blend are shown in Table 16.

TABLE 16
Prototype In-Process Blend Uniformity Results

| | % Potency | | | | | |
|---|---|---|---|---|---|---|
| | Lead Formulation | | Alternate 1 | | Altemate2 | |
| | 2 mg | 10 mg | 2 mg | 10 mg | 2 mg | 10 mg |
| Sample No. | Blend | Blend | Blend | Blend | Blend | Blend |
| 1 | 101.1 | 99.8 | 100.8 | 98.8 | 100.5 | 98.0 |
| 2 | 106.9 | 98.3 | 97.9 | 101.1 | 102.6 | 101.0 |
| 3 | 98.5 | 99.8 | 99.5 | 100.1 | 101.3 | 99.7 |
| Mean | 102.2 | 99.3 | 99.4 | 100.0 | 101.5 | 99.5 |
| ¾RSD | 4.2 | 0.9 | 1.4 | 1.1 | 1.0 | 1.5 |

Once it was confirmed that all blends passed typical in-process blend uniformity specifications (each individual sample % Potency 90.0-110.0%, mean % Potency 95.0-105.0%, and % RSD of the potency for all samples ≤5.0), the blends were used to prepare tablets on a rotary tablet press. Tablets were produced using 0.240×0.415 inch, scored, caplet-shaped, flat tooling (see Appendix III for tooling diagram) targeting a total tablet weight of 150 mg. Scored, caplet-shaped tooling was selected with the intention of having the ability to break the 2 mg and 10 mg ODTs in half to dose 1 mg and 5 mg, respectively. Each batch of tablets was assessed for Content Uniformity as outlined in USP <905>. All prototype ODT batches met USP <905> requirements for Content Uniformity (Acceptance Value, AV≤15.0). Content Uniformity results for each batch of tablets are shown in Table 17.

TABLE 17
Prototype Content Uniformity Results

| | % Potency | | | | | |
|---|---|---|---|---|---|---|
| | Lead Formulation | | Alternate 1 | | Alternate 2 | |
| Sample No. | 2 mg | 10 mg | 2 mg | 10 mg | 2 mg | 10 mg |
| 1 | 99.2 | 101.1 | 100.5 | 96.8 | 102.9 | 101.4 |
| 2 | 98.9 | 99.1 | 95.3 | 96.6 | 90.3 | 101.1 |
| 3 | 101.6 | 94.6 | 97.0 | 99.9 | 103.0 | 97.1 |
| 4 | 94.6 | 98.5 | 99.7 | 96.2 | 97.1 | 99.4 |
| 5 | 90.1 | 96.4 | 94.2 | 95.7 | 96.5 | 95.6 |
| 6 | 94.2 | 97.0 | 93.4 | 99.1 | 94.7 | 98.5 |
| 7 | 98.6 | 100.0 | 100.0 | 100.7 | 95.4 | 99.0 |
| 8 | 99.0 | 98.6 | 97.7 | 95.7 | 96.9 | 94.3 |
| 9 | 102.6 | 101.6 | 95.1 | 95.8 | 107.8 | 98.7 |
| 10 | 100.3 | 100.1 | 100.2 | 97.4 | 104.9 | 98.1 |
| Mean | 97.9 | 98.7 | 97.3 | 97.4 | 99.0 | 98.3 |
| SD | 3.8 | 2.2 | 2.7 | 1.9 | 5.4 | 2.2 |
| AV | 9.7 | 5.3 | 7.7 | 5.7 | 13.011 | 5.5 |

^{#}AV < 10 when theoretical concentration 1 s normalized for tablet weight of each sample.

### ODT Prototype Stability

Once ODTs were set aside for initial testing, the remaining tablets were packaged in two configurations for stability testing. Tablets were packaged in blister packs in a 2×5 array or in an induction-sealed 60 cc HDPE bottle containing 25-30 ODTs and a single 1-gram Sorbit desiccant canister. The ODTs were placed on stability at 25° C./60% RH and 40° C./75% RH. The stability data for dosage strength, packaging configuration, and storage condition are provided in EXAMPLE 2.

### Excipient Screen on Analytical Method

In parallel with the initial formulation development work, excipients were screened on the HPLC analytical method to determine if they would interfere significantly with the R-baclofen main peak or other related species peaks in the chromatography. Approximately 1 to 2 mg/mL solutions of the excipients in the method diluent (85% 50 mM Potassium Phosphate, pH 3.0, 15% ACN) were prepared, filtered, and analyzed by HPLC. The solubility of each excipient is noted in Table 18.

TABLE 18
Excipients Screened on HPLC Method

| | Completely Soluble |
|---|---|
| | in 85% 50 mM |
| | Potassium Phosphate, |
| Excipient | pH 3.0, 15% ACN |
| Lubripharm | No |
| Pharmaburst 500 | No |
| F-MELT | No |
| Pearlitol 300DC | Yes |
| Polyplasdone XL | No |
| Sucralose | Yes |
| Pruv | No |
| N-C Strawberry Flavor | Yes |
| N-C Orange Flavor | No |
| Masking Agent | No |
| RXcipients FM1000 | No |

When tested in chromatography for the 1-2 mg/mL excipient solutions, a blank solution, and a reference standard solution, most excipients did not produce significant peaks in the chromatography. Significant peaks were seen in the chromatography obtained for the 1-2 mg/mL solutions of N—C Strawberry Flavor, N—C Orange Flavor, and Masking Agent. These solutions were diluted to approximately 50 μg/mL (closer to the concentration that would be seen during assay analysis of the ODT) and reanalyzed. No significant peaks were seen in the chromatography of the diluted samples.

### Conclusions

A R-baclofen ODT formulation with excellent disintegration time and taste masking profiles was successfully developed. Multiple formulations met the disintegration requirements, but Formulation 15 combined fast disintegration with a good taste and mouth feel. Direct blending with a mid-stream sieving step was successful in producing uniform blends at the 2 mg and 10 mg dosage strengths. Direct compression of the uniform blends produced tables with acceptable content uniformity as required by USP <905>.

Tablet compression and tablet hardness were important factors in optimizing tablet disintegration. ODTs with a hardness of 2-3 kp combined quick disintegration with minimal weight loss during friablity testing. Prototype stability showed that it is necessary to take precautions to minimize ODT moisture uptake. A significant amount of moisture uptake was associated with reduced tablet hardness and an increase in weight loss (and tablet breaking) during friability testing. The SWT test provided a useful means to compare formulations since it was sensitive to changes in formulation and production process (e.g., increase in compression force). The SWT test results also correlated well with in vivo disintegration time and, thus, served as a useful in vitro test used to predict in vivo performance.

### Example 2

### Orally Disintegrating Tablets (ODTs) and Additional Stability Testing

Orally disintegrating tablets (ODTs) were manufactured as white, round, orally disintegrating tablets with flat-face, bevel-edged, plain-faced, and 150 mg total weight.

TABLE 19
ODT debossed dimensions

| | Dose | Figure Debossed Width (inches) | | | |
|---|---|---|---|---|---|
| Shape | Strength | S | 2 | 0 | 5 |
| bevel-edge arc | 5 mg | 0.0378 | 0.0376 | 0.0386 | 0.0395 |
| triangle | (S205) | | | | |
| bevel-edged | 10 mg | 0.0343 | 0.0342 | 0.0157 | 0.0351 |
| square | (S210) | | | | |
| flat-face | 15 mg | 0.0350 | 0.0350 | 0.0160 | 0.0350 |
| bevel-edged | (S215) | | | | |
| round | | | | | |

The spatial dimensions debossed on the ODTs are presented in Table 20.

TABLE 20
ODT spatial dimensions

| | Dose | Distance between Figures (inches) | | |
|---|---|---|---|---|
| Shape | Strength | S and 2 | 2 and 0 | 0 and 5 |
| bevel-edge arc | 5 mg | 0.0111 | 0.0116 | 0.0108 |
| triangle | (S205) | | | |
| | | S and 2 | 2 and 1 | 1 and 0 |
| bevel-edged | 10 mg | 0.0101 | 0.0116 | 0.0100 |
| square | (S210) | | | |
| | | S and 2 | 2 | 1 |
| flat-face | 15 mg | 0.0100 | 0.0100 | 0.0100 |
| bevel-edged | (S215) | | | |
| round | | | | |

ODT were tested at three dose strengths (5 mg, 10 mg, and 15 mg) distinguished by different shapes and nomenclature. The shapes of the ODTs are as follows: the 5 mg dose strength as a bevel-edge arc triangle tablet, debossed with S205; the 10 mg dose strength as a bevel-edged square tablet, debossed with S210 and the 15 mg dose strength as a flat-face bevel-edged round tablet, debossed with S215. All tablets will be 150 mg total weight.

Given R-baclofen is a single enantiomer of racemic baclofen, both specific rotation and optical purity (by capillary electrophoresis for determination of percent S-isomer) were added as attributes to monitor possible conversion of the isomer.

Four cGMP (Current Good Manufacturing Practice) batches (batches 0712741, 0802672, 0911218 and 1000411) R-baclofen were placed on long-term (25° C.±2° C./60% RH) and accelerated (40° C.±2° C./75% RH) stability following International Conference on Harmonization (ICH) guidelines. Data at 25° C.±2° C./60% RH has been collected for 48, 36, and 18 months for batch 0712741, 0802672, and 0911218/1000411, respectively. Data at 40° C.±2° C./75% RH has been collected for 6 months in all four batches.

All attributes tested were within specification. No unusual observations were identified. Chemical stability showed no significant difference between 25° C.±2° C./60% RH and 40° C.±2° C./75% RH. A slight increase in impurity 4-(4-chlorophenyl) pyrrolidin-2-could be observed at 25° C.±2° C./60% RH (Table 21) and 40° C.±2° C./75% RH (Table 22), but are within the specification limit of not more than 0.50%.

TABLE 21
4-(4-chlorophenyl)-2-pyrrolidin-2-one Quantity (Percent)
at 25° C. ± 2° C./60% RH1

| | Duration (months) | | | | | | | | | |
|---|---|---|---|---|---|---|---|---|---|---|
| Batch | 0 | 3 | 6 | 9 | 12 | 18 | 24 | 36 | 48 | Increase |
| 0712741 | 0.06 | 0.05 | 0.06 | 0.06 | 0.07 | 0.08 | 0.09 | 0.11 | 0.13 | 0.07 |
| 0802672 | 0.11 | 0.12 | 0.12 | 0.13 | 0.14 | 0.18 | 0.16 | 0.18 | | 0.07 |
| 0911218 | 0.16 | 0.18 | 0.19 | 0.19 | 0.19 | 0.21 | | | | 0.05 |
| 1000411 | 0.02 | 0.03 | 0.04 | 0.04 | 0.05 | 0.06 | | | | 0.04 |

1Quantity determined by HPLC

TABLE 22
4-(4-chlorophenyl)-2-pyrrolidin-2-one Quantity (Percent)
at 40° C. ± 2° C./75% RH1

| | Duration (months) | | | |
|---|---|---|---|---|
| Batch | 0 | 3 | 6 | Increase |
| 0712741 | 0.06 | 0.09 | 0.13 | 0.07 |
| 0802672 | 0.11 | 0.16 | 0.20 | 0.07 |
| 0911218 | 0.16 | 0.20 | 0.24 | 0.05 |
| 1000411 | 0.02 | 0.06 | 0.10 | 0.04 |

¹Quantity determined by HPLC

Thus far batches 0712741, 0802672, 0911218 and 1000411 have been tested using the original attributes for R-baclofen as presented in Table 23.

TABLE 23
Stability Data for Arbaclofen Orally Disintegrating Tablet, 5 mg, Lot# 16148 at 5° C.

| Test | Acceptance Criteria | | 52 Months | | | | 64 Months | | | |
|---|---|---|---|---|---|---|---|---|---|---|
| Appearance | White uncoated round tablet | | Conforms | | | | Conforms | | | |
| Assay | 90.0-110.0% of Label Claim | | 97.3% | | | | 96.5% | | | |
| Water Content | NMT 10.0% | | 2.3% | | | | 2.1% | | | |
| Disintegration | NMT 30 s | | Min: 3 s | | | | Min: 3 s | | | |
| | | | Max: 4 s | | | | Max: 3 s | | | |
| | | | Average: 3 s | | | | Average: 3 s | | | |
| Dissolution | Report results | Time (min) | 1 | 5 | 10 | 30 | 1 | 5 | 10 | 30 |
| | at 1, 5, 10, | Min | 90 | 101 | 99 | 99 | 78 | 93 | 94 | 94 |
| | and 30 min | Max | 99 | 104 | 104 | 104 | 92 | 101 | 102 | 102 |
| | | Average | 95 | 102 | 102 | 102 | 85 | 96 | 97 | 97 |
| | | % RSD | 4.7 | 1.1 | 1.6 | 1.9 | 5.2 | 3.4 | 3.1 | 3.1 |

| Related | Baclofen lactam NMT 2.0% | 0.31% | 0.6% |
|---|---|---|---|
| Substances | Other individual degradation | RRT 1.80 0.06% | RRT 1.818 0.1% |
| | products NMT 0.2% | RRT 1.92 0.06% | RRT 1.935 0.1% |
| | Total degradation | 0.43% | 0.7% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

Note:
NMT: Not more than

TABLE 24
Stability Data for Arbaclofen Orally Disintegrating Tablet, 5 mg, Lot# 16148 at 25° C./60% RH

| Test | Acceptance Criteria | | 52 Months | | | | 64 Months | | | |
|---|---|---|---|---|---|---|---|---|---|---|
| Appearance | White uncoated round tablet | | Conforms | | | | Conforms | | | |
| Assay | 90.0-110.0% of Label Claim | | 95.8% | | | | 95.7% | | | |
| Water Content | NMT 10.0% | | 2.1% | | | | 1.7% | | | |
| Disintegration | NMT 30 s | | Min: 3 s | | | | Min: 1 s | | | |
| | | | Max: 5 s | | | | Max: 1 s | | | |
| | | | Average: 4 s | | | | Average: 1 s | | | |
| Dissolution | Report results | Time (min) | 1 | 5 | 10 | 30 | 1 | 5 | 10 | 30 |
| | at 1, 5, 10, | Min | 83 | 95 | 96 | 96 | 86 | 93 | 93 | 93 |
| | and 30 min | Max | 89 | 99 | 99 | 99 | 92 | 100 | 101 | 102 |
| | | Average | 85 | 97 | 97 | 97 | 89 | 96 | 96 | 97 |
| | | % RSD | 2.6 | 1.7 | 1.3 | 1.3 | 2.9 | 2.3 | 2.8 | 3.0 |

| Related | Baclofen lactam NMT 2.0% | 0.96% | 0.8% |
|---|---|---|---|
| Substances | Other individual degradation | RRT 1.80 0.06% | RRT 1.818 0.1% |
| | products NMT 0.2% | | RRT 1.935 0.1% |
| | Total degradation | 1.0% | 0.9% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 25
Stability Data for Arbaclofen Orally Disintegrating Tablet, 10 mg, Lot# 16160 at 5° C.

| Test | Acceptance Criteria | | 52 Months | | | | 64 Months | | | |
|---|---|---|---|---|---|---|---|---|---|---|
| Appearance | White uncoated round tablet | | Conforms | | | | Conforms | | | |
| Assay | 90.0-110.0% of Label Claim | | 97.2% | | | | 98.6% | | | |
| Water Content | NMT 10.0% | | 2.0% | | | | 1.4% | | | |
| Disintegration | NMT 30 s | | Min: 3 s | | | | Min: 3 s | | | |
| | | | Max: 5 s | | | | Max: 3 s | | | |
| | | | Average: 4 s | | | | Average: 3 s | | | |
| Dissolution | Report results | Time (min) | 1 | 5 | 10 | 30 | 1 | 5 | 10 | 30 |
| | at 1, 5, 10, | Min | 91 | 101 | 100 | 101 | 89 | 100 | 101 | 100 |
| | and 30 min | Max | 96 | 103 | 103 | 103 | 97 | 102 | 103 | 103 |
| | | Average | 94 | 102 | 102 | 102 | 93 | 102 | 102 | 102 |
| | | % RSD | 1.9 | 0.6 | 1.0 | 1.0 | 2.7 | 1.1 | 0.9 | 1.3 |

| Related | Baclofen lactam NMT 2.0% | 0.29% | 0.6% |
|---|---|---|---|
| Substances | Other individual degradation | RRT 1.80 0.06% | RRT 1.818 0.1% |
| | products NMT 0.2% | RRT 1.92 0.05% | RRT 1.935 0.1% |
| | Total degradation | 0.40% | 0.7% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 26
Stability Data for Arbaclofen Orally Disintegrating Tablet, 10 mg, Lot# 16160 at 25° C./60% RH

| Test | Acceptance Criteria | | 52 Months | | | | 64 Months | | | |
|---|---|---|---|---|---|---|---|---|---|---|
| Appearance | White uncoated round tablet | | Conforms | | | | Conforms | | | |
| Assay | 90.0-110.0% of Label Claim | | 96.8% | | | | 98.2% | | | |
| Water Content | NMT 10.0% | | 2.0% | | | | 2.2% | | | |
| Disintegration | NMT 30 s | | Min: 3 s | | | | Min: 2 s | | | |
| | | | Max: 4 s | | | | Max: 2 s | | | |
| | | | Average: 4 s | | | | Average: 2 s | | | |
| Dissolution | Report results | Time (min) | 1 | 5 | 10 | 30 | 1 | 5 | 10 | 30 |
| | at 1, 5, 10, | Min | 87 | 100 | 100 | 100 | 84 | 95 | 96 | 97 |
| | and 30 min | Max | 94 | 101 | 101 | 102 | 91 | 98 | 99 | 100 |
| | | Average | 91 | 100 | 100 | 101 | 88 | 97 | 98 | 98 |
| | | % RSD | 2.8 | 0.5 | 0.6 | 0.6 | 2.9 | 1.4 | 1.3 | 1.2 |

| Related | Baclofen lactam NMT 2.0% | 0.84% | 0.7% |
|---|---|---|---|
| Substances | Other individual degradation | RRT 0.29 0.05% | RRT 1.818 0.1% |
| | products NMT 0.2% | RRT 1.80 0.06% | RRT 1.935 0.1% |
| | | RRT 1.92 0.05% | |
| | Total degradation | 1.0% | 0.8% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 27
Stability Data for Arbaclofen Orally Disintegrating Tablet, 15 mg, Lot# 16175 at 5° C.

| Test | Acceptance Criteria | | 52 Months | | | | 63 Months | | | |
|---|---|---|---|---|---|---|---|---|---|---|
| Appearance | White uncoated round tablet | | Conforms | | | | Conforms | | | |
| Assay | 90.0-110.0% of Label Claim | | 98.0% | | | | 98.1% | | | |
| Water Content | NMT 10.0% | | 1.8% | | | | 1.9% | | | |
| Disintegration | NMT 30 s | | Min: 3 s | | | | Min: 3 s | | | |
| | | | Max: 3 s | | | | Max: 3 s | | | |
| | | | Average: 3 s | | | | Average: 3 s | | | |
| Dissolution | Report results | Time (min) | 1 | 5 | 10 | 30 | 1 | 5 | 10 | 30 |
| | at 1, 5, 10, | Min | 88 | 97 | 97 | 97 | 87 | 96 | 96 | 96 |
| | and 30 min | Max | 95 | 102 | 102 | 102 | 95 | 99 | 99 | 99 |
| | | Average | 92 | 100 | 100 | 99 | 92 | 97 | 98 | 98 |
| | | % RSD | 3.5 | 1.9 | 2.1 | 2.1 | 3.5 | 1.3 | 1.2 | 1.2 |

| Related | Baclofen lactam NMT 2.0% | 0.26% | 0.5% |
|---|---|---|---|
| Substances | Other individual degradation | RRT 0.29 0.07% | <LOQ |
| | products NMT 0.2% | | |
| | Total degradation | 0.33% | 0.5% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 28
Stability Data for Arbaclofen Orally Disintegrating Tablet, 15 mg, Lot# 16175 at 25° C./60% RH

| Test | Acceptance Criteria | | 52 Months | | | | 63 Months | | | |
|---|---|---|---|---|---|---|---|---|---|---|
| Appearance | White uncoated round tablet | | Conforms | | | | Conforms | | | |
| Assay | 90.0-110.0% of Label Claim | | 97.4% | | | | 97.6% | | | |
| Water Content | NMT 10.0% | | 2.4% | | | | 2.4% | | | |
| Disintegration | NMT 30 s | | Min: 3 s | | | | Min: 3 s | | | |
| | | | Max: 5 s | | | | Max: 3 s | | | |
| | | | Average: 4 s | | | | Average: 3 s | | | |
| Dissolution | Report results | Time (min) | 1 | 5 | 10 | 30 | 1 | 5 | 10 | 30 |
| | at 1, 5, 10, | Min | 86 | 97 | 98 | 98 | 89 | 96 | 97 | 97 |
| | and 30 min | Max | 90 | 100 | 101 | 101 | 94 | 100 | 100 | 100 |
| | | Average | 88 | 98 | 99 | 99 | 91 | 98 | 99 | 99 |
| | | % RSD | 1.8 | 1.2 | 1.5 | 1.7 | 2.4 | 1.4 | 1.3 | 1.3 |

| Related | Baclofen lactam NMT 2.0% | 0.75% | 0.6% |
|---|---|---|---|
| Substances | Other individual degradation | <LOQ | <LOQ |
| | products NMT 0.2% | | |
| | Total degradation | 0.75% | 0.6% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 29
Stability Data for Arbaclofen Orally Disintegrating Tablet, 20 mg, Lot# 17011 at 5° C.

| Test | Acceptance Criteria | | 52 Months | | | | 62 Months | | | |
|---|---|---|---|---|---|---|---|---|---|---|
| Appearance | White uncoated round tablet | | Conforms | | | | Conforms | | | |
| Assay | 90.0-110.0% of Label Claim | | 98.0% | | | | 98.2% | | | |
| Water Content | NMT 10.0% | | 2.1% | | | | 2.0% | | | |
| Disintegration | NMT 30 s | | Min: 3 s | | | | Min: 3 s | | | |
| | | | Max: 4 s | | | | Max: 3 s | | | |
| | | | Average: 3 s | | | | Average: 3 s | | | |
| Dissolution | Report results | Time (min) | 1 | 5 | 10 | 30 | 1 | 5 | 10 | 30 |
| | at 1, 5, 10, | Min | 88 | 99 | 100 | 100 | 83 | 96 | 97 | 97 |
| | and 30 min | Max | 94 | 101 | 102 | 102 | 97 | 102 | 102 | 102 |
| | | Average | 91 | 100 | 100 | 101 | 90 | 98 | 99 | 99 |
| | | % RSD | 3.1 | 0.6 | 0.7 | 0.8 | 4.9 | 1.9 | 1.6 | 1.6 |

| Related | Baclofen lactam NMT 2.0% | 0.25% | 0.5% |
|---|---|---|---|
| Substances | Other individual degradation | RRT 1.80 0.06% | RRT 1.818 0.1% |
| | products NMT 0.2% | RRT 1.92 0.06% | RRT 1.935 0.1% |
| | Total degradation | 0.37% | 0.6% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

TABLE 30
Stability Data for Arbaclofen Orally Disintegrating Tablet, 20 mg, Lot# 17011 at 25° C./60% RH

| Test | Acceptance Criteria | | 52 Months | | | | 62 Months | | | |
|---|---|---|---|---|---|---|---|---|---|---|
| Appearance | White uncoated round tablet | | Conforms | | | | Conforms | | | |
| Assay | 90.0-110.0% of Label Claim | | 98.4% | | | | 98.1% | | | |
| Water Content | NMT 10.0% | | 2.0% | | | | 1.5% | | | |
| Disintegration | NMT 30 s | | Min: 3 s | | | | Min: 2 s | | | |
| | | | Max: 3 s | | | | Max: 2 s | | | |
| | | | Average: 3 s | | | | Average: 2 s | | | |
| Dissolution | Report results | Time (min) | 1 | 5 | 10 | 30 | 1 | 5 | 10 | 30 |
| | at 1, 5, 10, | Min | 86 | 100 | 100 | 100 | 82 | 96 | 96 | 97 |
| | and 30 min | Max | 93 | 100 | 100 | 100 | 94 | 100 | 100 | 100 |
| | | Average | 90 | 100 | 100 | 100 | 89 | 98 | 98 | 98 |
| | | % RSD | 3.2 | 0.1 | 0.1 | 0.2 | 5.2 | 1.5 | 1.1 | 1.1 |

| Related | Baclofen lactam NMT 2.0% | 0.72% | 0.6% |
|---|---|---|---|
| Substances | Other individual degradation | RRT 0.29 0.07% | RRT 1.818 0.1% |
| | products NMT 0.2% | RRT 1.80 0.06% | RRT 1.935 0.1% |
| | | RRT 1.92 0.05% | |
| | Total degradation | 0.90% | 0.7% |
| | products NMT 3.0% | | |
| Microbiological | TAMC: NMT 1000 cfu/g | Not Applicable | <10 cfu/g |
| Attributes | TYMC: NMT 100 cfu/g | Not Applicable | <10 cfu/g |
| | E. coli: Absent/1 g | Not Applicable | Absent/1 g |
| | S. aureus: Absent/1 g | Not Applicable | Absent/1 g |
| | P. aeruginosa: Absent/1 g | Not Applicable | Absent/1 g |
| | Bile-tolerant gram negative: | Not Applicable | Absent/1 g |
| | Absent/1 g | | |

| | | |
|---|---|---|
| Salmonella: Absent/10 g | Not Applicable | Absent/10 g |

### Example 3

### Comparison of Film/Foil, Foil/Foil, and Bottle Packaging Configurations Product Stability

A 24-month test interval was used for R-baclofen ODT (5 mg, 10 mg, and 15 mg dose strengths) packaged in film/foil and stored at room temperature based on the stability data available to date from 25° C./60% RH storage. No out-of-specification results have been observed for any dose strength or packaging configuration stored at 25° C./60% RH. One known specified impurity, 4-(4-chlorophenyl) pyrrolidin-2-one, demonstrates an increase with time, but will not reach an unacceptable level (more than 2.0%) until at least 62 months.

Data in the film/foil packaging configuration is limited to one lot of ODT at a dose strength of 5 mg. To evaluate the feasibility of using data collected in other packaging configurations and other dose strengths for predicting the stability of all commercial dose strengths in film/foil, the following two data sets are presented.
(a) Comparison of Film/Foil, Foil/Foil, and Bottle Packaging Configurations Product Stability: The data was collected for one lot of ODT packaged into three configurations at 25° C./60% RH and 40° C./75% RH. The data indicates that the three packaging configurations give similar product stability at each condition.
(b) Comparison of 5 mg, 10 mg, and 15 mg ODT Stability: The data was collected for multiple lots of clinical ODT at the intended commercial dose strengths stored at 25° C./60% RH. The data indicates the increase in 4-(4-chlorophenyl) pyrrolidin-2-one proceeds in a predictable linear fashion independent of the dose strength.

Together, these data sets provide confidence that historical stability data can be applied to predict the stability of the commercial product.

Lot 442519 of ODT at the 5 mg dose strength was packaged into three configurations: film/foil blister, foil/foil blister and high-density polyethylene wide mouth round containers (bottle). Table 31 provides a summary of the packaging materials used for each packaging configuration.

TABLE 31
Summary of Packaging Materials by Packaging Configuration

| Configuration | Component | Description |
|---|---|---|
| Film/Foil | Blister Film | 250 μm PVC, 50 um polychlorotrifluoroethylene |
| | | (Tekni-Films USA) |
| Blister | Foil Lid | 25 μm push-through aluminum foil with heatseal coating |
| | | LX4DP consisting of polyvinyl chloride/polyvinyl acetate |
| | | copolymer (Constantia Hueck Foils) |
| Foil/Foil | Blister Foil | 25 μm oriented polyamide film, NST 14 adhesive lacquer, |
| Blister | (cold | 45 μm aluminum foil, NST 14 adhesive lacquer, 60 μm |
| | formable) | polyvinyl chloride (Constantia Hueck Foils) |
| | Foil Lid | 25 μm push-through aluminum foil with heatseal coating |
| | | LX4DP consisting of PVC/PVAC copolymer |
| | | (Constantia Hueck Foils) |
| Bottle | Bottle | 150 cc white opaque high density polyethylene wide mouth |
| (100 count) | | round bottle (Drug Plastics & Glass Company, Inc.) |
| | | (Bottle Resin: Phillips Marlex HHM 5502 BN) |
| | Cap | 38 mm-400 two piece SecuRx ® CRC |
| | | Closure Material: Polypropylene inner and outer shells |
| | | (Closure Resin: Marlex ® HGL-200 or INEOS H20E-20) |
| | | Liner: Foilseal ™, Selig 0.035″ Pup FSM-1 (Drug Plastics |
| | | Closures) |
| | Other | 9 gram polyester coil (Carolina Absorbant Cotton) |
| | | 1.0 gram silica gel canister, 1 per bottle (Multisorb |
| | | Technologies, Inc.) |

Data acquired through the 24-month interval at 25° C./60% RH in each packaging configuration are not significantly different. Table 32 and Table 33 provide a summary of the stability data collected in each packaging configuration stored at 25° C./60% RH for description, assay, trending impurity 4-(4-chlorophenyl) pyrrolidin-2-one (CAS 22518-27-0, also known as related compound A and baclofen lactam), total related substances, and water. Data acquired at 40° C./75% RH has also been included. Though an out-of-specification result at this storage condition was obtained for the attribute Description, all other attributes remain within specification and support the equivalence of the three packaging configurations. The out-of-specification result for Description was investigated and found to be due to the discoloration of sucralose.

TABLE 32
Summary of Description Attribute Data by Packaging Configuration

| | Description: lot 442519, 5 mg ODT | | | | | |
|---|---|---|---|---|---|---|
| Time point | 25° C./60% RH | | | 40° C./75% RH | | |
| (month) | Film/Foil | Foil/Foil | Bottle | Film/Foil | Foil/Foil | Bottle |
| Release | meets | meets | meets | meets | meets | meets |
| 0 | meets | meets | meets | meets | meets | meets |
| 1 | meets | meets | meets | meets | meets | meets |
| 2 | meets | meets | meets | meets | meets | Does not meet |
| 3 | meets | meets | meets | meets | meets | Does not meet |
| 6 | meets | meets | meets | meets | Does not meet | Does not meet |
| 9 | meets | meets | meets | | | |
| 12 | meets | meets | meets | | | |
| 18 | meets | meets | meets | | | |
| 24 | meets | meets | meets | | | |

TABLE 33
Summary of Assay Attribute Data by Packaging Configuration#

| | R-baclofen Assay (%): lot 442519, 5 mg ODT | | | | | |
|---|---|---|---|---|---|---|
| Time point | 25° C./60% RH | | | 40° C./75% RH | | |
| (month) | Film/Foil | Foil/Foil | Bottle | Film/Foil | Foil/Foil | Bottle |
| Release | 94.6 | 94.6 | 94.6 | 94.6 | 94.6 | 94.6 |
| 0 | 97.8 | 97.5 | 94.6 | 97.8 | 97.5 | 94.6 |
| 1 | 96.7 | 98.4 | 97.3 | 97.0 | 97.2 | 96.4 |
| 2 | 97.4 | 97.0 | 97.3 | 96.1 | 96.9 | 97.5 |
| 3 | 97.9 | 97.5 | 96.7 | 97.1 | 97.0 | 96.9 |
| 6 | 97.0 | 97.6 | 96.9 | 95.0 | 95.4 | 96.0 |
| 9 | 97.0 | 97.1 | 96.5 | | | |
| 12 | 97.6 | 97.5 | 97.4 | | | |
| 18 | 96.3 | 97.0 | 97.4 | | | |
| 24 | 97.1 | 96.5 | 96.5 | | | |

^{#}Tentative specification: 90.0-110.0%

### Example 4

### Segregation Tests for Baclofen Formulations

Segregation tests were conducted on a representative baclofen composition described in Table 34 below to evaluate the potential for the material to segregate by the fluidization and sifting mechanisms using Material Sparing Segregation Testers (Jenike & Johanson). After these tests, samples were collected from different points in the testers. These samples were also analyzed for panicle size distribution (using laser diffraction). A low feed pressure of 0.5 bar was used for the panicle size tests to minimize the attrition of fragile particles.

TABLE 34
Exemplary R-baclofen composition

| | wt % by |
|---|---|
| | weight of the |
| Component | composition |
| R-baclofen | 6.7 |
| F-MELT | 83.9 |
| Crospovidone | 5 |
| Sucralose | 2 |
| Sodium stearyl fumarate | 2 |
| Stawberry flavor | 0.3 |
| Masking flavor | 0.1 |

### Fluidization Segregation

Fluidization can cause vertical segregation, i.e., horizontal layers of fines and coarse material. Fine powders generally have a lower permeability than coarse materials and therefore tend to retain ah longer. Thus, when a hopper is being filled, the coarse particles are driven into the bed while the fine particles remain fluidized near the surface. This can also occur after tumble blending if the material is fluidized during blending. Air entrainment often develops in materials that contain a significant percentage of panicles below 100 microns in size. Fluidization segregation is likely to occur when fine materials are pneumatically conveyed, filled or discharged at high rates, or if gas counter-flow is present.

The fluidization segregation test was run by first fluidizing a column of material by injecting ah at the bottom. A specific test profile (air flow rates and times) was used to get the material fluidized. The column was then split into (typically) sixteen equal sections. If fluidization segregation takes place, samples from the bottom will be coarser (and/or have heavier panicles) than samples from the top.

A single fluidization segregation test was run on the R-baclofen blends. Odd layers were used for particle size analysis, and even layers were used for assays. At the peak flow rate (e.g., 3.5 or 3.0 cm/s) of each test, the average bed density was about 0.35 g/cm³.

Based on the test results, it was found that each blend's tendency to segregate by the fluidization segregation mechanism is low with respect to particle size.

### Sifting Segregation

Sifting is a process by which smaller particles move through a matrix of larger ones. It is the most common method of segregation. Four conditions must exist for sifting to occur:
(1) A difference in particle size between the individual components. This ratio can be as low as 1.3 to 1. In general, the larger the ratio of particle sizes, the greater the tendency for particles to segregate by sifting.
(2) A sufficiently large mean particle size. Sifting segregation can occur with a mean particle size in the 50 micron range and can become a dominant segregation mechanism if the mean particle size is above 100 microns.
(3) Free flowing material. This allows the smaller particles to sift through the matrix of larger particles. With cohesive materials, the fine particles are bound to one another and do not enter the voids among the coarse particles.
(4) Interparticle motion. This can be caused during formation of a pile, by vibration, or by a velocity gradient across the flowing material.

All four of these conditions must exist for sifting segregation to occur. If any one of these conditions does not exist, the material will not segregate by this mechanism. In materials having a range of particle sizes, sifting segregation may significantly impact product quality and handleability. As an illustration of the segregation mechanism, consider a pile formed by a falling stream of material. If sifting segregation takes place, the finer particles will tend to sift through the larger particles and concentrate under the point of impact. As the concentration of fines builds up in the pile center, the coarser particles will tend to roll or slide to the edges of this pile.

The sifting segregation test was performed by filling a narrow (quasi-2D) test cell to one side, allowing a pile to form within the cell. If a material segregates by sifting, the coarse particles will flow to the far side, away from the fill point, while fines will accumulate under the fill point. After the cell was filled, samples were extracted from the bottom of the tester, in layers, with a given layer subdivided into five slots. The first layer extracted was designated layer number one. And anywhere from 9 to 12 layers can be collected, depending on the surcharge angle of the material. Slot were designated A through E. with A being away from the fill point, and E under the fill point.

If sifting segregation takes place. “A” samples (from all layers) will be coarser than “E” samples. A single test was run on the blends. All samples were collected. Samples A, C, and E were analyzed via laser diffraction for particle size.

Based on the test results, it was found that each blend's tendency to segregate by a sifting mechanism is low with respect to particle size.

The particle size distribution is summarized in Table 35.

TABLE 35
Particle Size Distribution

| Sample | A | B | C | D | E | F |
|---|---|---|---|---|---|---|
| Volume Weighted | 131.8 | 121.8 | 129.6 | 128.2 | 129.3 | 129.03 |
| Mean (μm) | | | | | | |
| Surface Weighted | 43 | 52.4 | 60.6 | 58.9 | 59.3 | 59.6 |
| Mean (μm) | | | | | | |
| D10^{&} (μm) | 19.4 | 25.7 | 30.1 | 29.0 | 29.2 | 29.4 |
| D50^{&} (μm) | 118.7 | 107.6 | 117.3 | 116.4 | 117.3 | 117.0 |
| D90^{&} (μm) | 259.2 | 237 | 245.0 | 242.3 | 244.7 | 244.0 |

^{&}D10, D50, and D90 are percentile values that indicate the size below which 10%, 50% or 90% of all particles are found.

The present disclosure is not to be limited in scope by the specific embodiments described herein. Indeed, various modifications of the invention in addition to those described herein will become apparent to those skilled in the art from the foregoing description and the accompanying FIGURES. Such modifications are intended to fall within the scope of the appended claims.

1. An orally disintegrating composition, comprising:
baclofen, a stereoisomer thereof, a derivative thereof, an analog thereof, a prodrug thereof, or a pharmaceutically acceptable salt thereof;
an excipient;
a disintegrating agent; and
a lubricating agent.

2. The composition of claim 1, wherein (a) the baclofen is R-baclofen, (b) the excipient comprises F-MELT, (c) the disintegrating agent comprises crospovidone, or (d) the lubricating agent comprises sodium stearyl fumarate.

3. (canceled)

4. The composition of claim 1, wherein the excipient comprises F-MELT type C.

5. (canceled)

6. (canceled)

7. The composition of claim 1, further comprising a sweetener.

8. The composition of claim 7 wherein the sweetener comprises sucralose.

9. The composition of claim 1, further comprising a flavoring agent, a masking agent, or a combination thereof.

10. The composition of claim 1, comprising R-baclofen, F-MELT, crospovidone, sucralose, sodium stearyl fumarate, a flavoring agent, and a masking agent.

11. The composition of claim 10, comprising:
from 1.197% w/w to 1.463% w/w of R-baclofen,
from 80.343% w/w to 98.197% w/w of F-MELT,
from 4.5% w/w to 5.5% w/w of crospovidone,
from 1.8% w/w to 2.2% w/w of sodium stearyl fumarate,
from 1.8% w/w to 2.2% w/w of sucralose,
from 0.27% w/w to 0.33% w/w of the flavoring agent, and
from 0.09% w/w to 0.11% w/w of the masking agent.

12. The composition of claim 10, comprising:
about 1.33% w/w of R-baclofen,
about 89.27% w/w of F-MELT,
about 5% w/w of crospovidone,
about 2% w/w of sodium stearyl fumarate,
about 2% w/w of sucralose,
about 0.3% w/w of the flavoring agent, and
from 0.1% w/w of the masking agent.

13. The composition of claim 10, comprising:
about 1.33% w/w of R-baclofen,
about 89.27% w/w of F-MELT type C,
about 5% w/w of crospovidone,
about 2% w/w of sodium stearyl fumarate,
about 2% w/w of sucralose,
about 0.3% w/w of the flavoring agent, and
from 0.1% w/w of the masking agent.

14. The composition of claim 10, comprising:
about 2 mg of R-baclofen,
about 133.9 mg of F-MELT type C,
about 7.5 mg of crospovidone,
about 3 mg of sodium stearyl fumarate,
about 3 mg of sucralose,
about 0.45 mg of the flavoring agent, and
about 0.15 mg of the masking agent.

15. The composition of claim 10, comprising:
from 3% w/w to 3.67% w/w of R-baclofen,
from 78.54% w/w to 95.99% w/w of F-MELT,
from 4.5% w/w to 5.5% w/w of crospovidone,
from 1.8% w/w to 2.2% w/w of sodium stearyl fumarate,
from 1.8% w/w to 2.2% w/w of sucralose,
from 0.27% w/w to 0.33% w/w of the flavoring agent, and
from 0.09% w/w to 0.11% w/w of the masking agent.

16. The composition of claim 10, comprising:
about 3.33% w/w of R-baclofen,
about 87.27% w/w of F-MELT,
about 5% w/w of crospovidone,
about 2% w/w of sodium stearyl fumarate,
about 2% w/w of sucralose,
about 0.3% w/w of the flavoring agent, and
about 0.1% w/w of the masking agent.

17. The composition of claim 10, comprising:
about 3.33% w/w of R-baclofen,
about 87.27% w/w of F-MELT type C,
about 5% w/w of crospovidone,
about 2% w/w of sodium stearyl fumarate,
about 2% w/w of sucralose,
about 0.3% w/w of the flavoring agent, and
about 0.1% w/w of the masking agent.

18. The composition of claim 10, comprising:
about 5 mg of R-baclofen,
about 130.9 mg of F-MELT type C,
about 7.5 mg of crospovidone,
about 3 mg of sodium stearyl fumarate,
about 3 mg of sucralose,
about 0.45 mg of the flavoring agent, and
about 0.15 mg of the masking agent.

19. The composition of claim 10, comprising:
from 6.0% w/w to 7.34% w/w of R-baclofen,
from 75.54% w/w to 92.32% w/w of F-MELT,
from 4.5% w/w to 5.5% w/w of crospovidone,
from 1.8% w/w to 2.2% w/w of sodium stearyl fumarate,
from 1.8% w/w to 2.2% w/w of sucralose,
from 0.27% w/w to 0.33% w/w of the flavoring agent, and
from 0.09% w/w to 0.11% w/w of the masking agent.

20. The composition of claim 10, comprising:
about 6.67% w/w of R-baclofen,
about 83.93% w/w of F-MELT,
about 5% w/w of crospovidone,
about 2% w/w of sodium stearyl fumarate,
about 2% w/w of sucralose,
about 0.3% w/w of the flavoring agent, and
about 0.1% w/w of the masking agent.

21. The composition of claim 10, comprising:
about 6.67% w/w of R-baclofen,
about 83.93% w/w of F-MELT type C,
about 5% w/w of crospovidone,
about 2% w/w of sodium stearyl fumarate,
about 2% w/w of sucralose,
about 0.3% w/w of the flavoring agent, and
about 0.1% w/w of the masking agent.

22. The composition of claim 10, comprising:
about 10 mg of R-baclofen,
about 125.9 mg of F-MELT type C,
about 7.5 mg of crospovidone,
about 3 mg of sodium stearyl fumarate,
about 3 mg of sucralose,
about 0.45 mg of the flavoring agent, and
about 0.15 mg of the masking agent.

23. The composition of claim 10, comprising:
from 9% w/w to 11% w/w of R-baclofen,
from 72.54% w/w to 88.66% w/w of F-MELT,
from 4.5% w/w to 5.5% w/w of crospovidone,
from 1.8% w/w to 2.2% w/w of sodium stearyl fumarate,
from 1.8% w/w to 2.2% w/w of sucralose,
from 0.27% w/w to 0.33% w/w of the flavoring agent, and
from 0.09% w/w to 0.11% w/w of the masking agent.

24. The composition of claim 10, comprising:
about 10% w/w of R-baclofen,
about 80.6% w/w of F-MELT,
about 5% w/w of crospovidone,
about 2% w/w of sodium stearyl fumarate,
about 2% w/w of sucralose,
about 0.3% w/w of the flavoring agent, and
about 0.1% w/w of the masking agent.

25. The composition of claim 10, comprising:
about 10% w/w of R-baclofen,
about 80.6% w/w of F-MELT type C,
about 5% w/w of crospovidone,
about 2% w/w of sodium stearyl fumarate,
about 2% w/w of sucralose,
about 0.3% w/w of the flavoring agent, and
about 0.1% w/w of the masking agent.

26. The composition of claim 10, comprising:
about 15 mg of R-baclofen,
about 120.9 mg of F-MELT type C,
about 7.5 mg of crospovidone,
about 3 mg of sodium stearyl fumarate,
about 3 mg of sucralose,
about 0.45 mg of the flavoring agent, and
about 0.15 mg of the masking agent.

27. The composition of claim 9, wherein the flavoring agent comprises a strawberry flavoring agent.

28. The composition of claim 9, wherein the masking agent comprises a second flavoring agent or a second sweetener.

29. The composition of claim 1, wherein the composition has one or more characteristics of:
disintegrating in less than 15 seconds upon admixing with the aqueous medium after storage for 64 months;
dissolving in less than 5 minutes upon admixing with the aqueous medium after storage for 64 months;
stable for at least 64 months under a storage condition at 25° C.±2° C./60% RH or at least 6 months under a storage condition at 40° C.±2° C./75% RH; and
generating less than 0.25% w/w of impurity 4-(4-chlorophenyl) pyrrolidin-2-one after storage at 25° C.±2° C./60% RH for 48 months or at 40° C.±2° C./75% RH for 6 months.

30. (canceled)

31. A unit dose comprising the composition of claim 1 provided in a form of a tablet.

32. (canceled)

33. The unit dose of claim 31, wherein (a) the tablet comprises 2 mg, 5 mg, 10 mg, or 15 mg of the baclofen, a stereoisomer thereof, a derivative thereof, an analog thereof, a prodrug thereof, or a pharmaceutically acceptable salt thereof, or (b) the tablet comprises 150 mg of the composition.

34. (canceled)

35. The unit dose of claim 31, further comprising a foil packaging encapsulating the composition.

36. A method for forming a composition according to claim 1, comprising:
(a) passing an active pharmaceutical ingredient through a 80 mesh screen, wherein the active pharmaceutical ingredient comprises baclofen, a stereoisomer thereof, a derivative thereof, an analog thereof, a prodrug thereof, or a pharmaceutically acceptable salt thereof;
(b) blending about 0.2 kg of the active pharmaceutical ingredient and about 0.681 kg of the excipient for about 10 minutes at about 25 rpm to obtain a first mixture;
(c) blending about 1.834 kg of the excipient for about 10 minutes at about 25 rpm;
(d) adding to the excipient of step (c) to the first mixture to obtain a second mixture, and passing the second mixture through a 50 mesh screen;
(e) blending the second mixture with about 0.3 kg of the disintegrating agent, about 0.12 kg of the sweetener, about 0.018 kg of the flavoring agent, and about 0.006 kg of the masking agent for about 20 minutes at about 25 rpm to obtain a third mixture; and
(f) passing the lubricating agent through a 30 mesh screen, and blending about 0.12 kg of the lubricating agent with the third mixture for about 5 minutes at about 25 rpm to obtain the composition.

37. (canceled)

38. The method of claim 36, wherein (a) the excipient comprises F-MELT type C, (b) the disintegrating agent comprises crospovidone, (c) the lubricating agent comprises sodium stearyl fumarate, or (d) the sweetener comprises sucralose.

39. (canceled)

40. (canceled)

41. (canceled)
